SEQUENCE LISTING (70 sequences)

SEQ ID NO: 1 - DNA, 23 bp, Artificial
  PLEKHS1 G590A_F
ccaaggctgggatgatctagaag

SEQ ID NO: 2 - DNA, 28 bp, Artificial
  PLEKHS1 G590A_R
agcatatctgcaaaattttccatttcca

SEQ ID NO: 3 - DNA, 20 bp, Artificial
  PLEKHS1 G590A_P
  misc_feature (14)..(14): n is G or A.
cttttttgcaattnaacaat

SEQ ID NO: 4 - DNA, 24 bp, Artificial
  PLEKHS1 C593T_F
ggctgggatgatctagaagctttt

SEQ ID NO: 5 - DNA, 27 bp, Artificial
  PLEKHS1 C593T_R
aagtgcccataacagaaatacagcata

SEQ ID NO: 6 - DNA, 20 bp, Artificial
  PLEKHS1 C593T_P
  misc_feature (7)..(7): n is C or T.
attgaanaattgcaaaattg

SEQ ID NO: 7 - DNA, 20 bp, Artificial
  TERT_F1
agtggattcgcgggcacaga

SEQ ID NO: 8 - DNA, 18 bp, Artificial
  TERT_R1
cagcgctgcctgaaactc

SEQ ID NO: 9 - DNA, 23 bp, Artificial
  TERT_F2
cacccgtcctgccccttcacctt

SEQ ID NO: 10 - DNA, 23 bp, Artificial
  TERT_R2
ggcttcccacgtgcgcagcagga

SEQ ID NO: 11 - DNA, 18 bp, Artificial
  TERT_F3
gtcctgccccttcacctt

SEQ ID NO: 12 - DNA, 18 bp, Artificial
  TERT_R3
cagcgctgcctgaaactc

SEQ ID NO: 13 - DNA, 23 bp, Artificial
  BRAF exon 15_F
tcataatgcttgctctgatagga

SEQ ID NO: 14 - DNA, 22 bp, Artificial
  BRAF exon 15_R
ggccaaaaatttaatcagtgga

SEQ ID NO: 15 - DNA, 18 bp, Artificial
  NRAS codon 61_F
ccccttaccctccacacc

SEQ ID NO: 16 - DNA, 24 bp, Artificial
  NRAS codon 61_R
gaggttaatatccgcaaatgactt

SEQ ID NO: 17 - DNA, 21 bp, Artificial
  NRAS codon 12,  13_F
cttgctggtgtgaaatgactg

SEQ ID NO: 18 - DNA, 20 bp, Artificial
  NRAS codon 12,  13_R
tccgacaagtgagagacagg

SEQ ID NO: 19 - DNA, 20 bp, Artificial
  HRAS codon 61_F
gtcctcctgcaggattccta

SEQ ID NO: 20 - DNA, 17 bp, Artificial
  HRAS codon 61_R
cggggttcacctgtact

SEQ ID NO: 21 - DNA, 19 bp, Artificial
  HRAS codon 12,  13_F
ctgaggagcgatgacggaa

SEQ ID NO: 22 - DNA, 20 bp, Artificial
  HRAS codon 12,  13_R
aggctcacctctatagtggg

SEQ ID NO: 23 - DNA, 22 bp, Artificial
  KRAS codon 61_F1
ggtgcactgtaataatccagac

SEQ ID NO: 24 - DNA, 21 bp, Artificial
  KRAS codon 61_R1
tgatttagtattatttatggc

SEQ ID NO: 25 - DNA, 24 bp, Artificial
  KRAS codon 61_F2
tgaagtaaaaggtgcactgtaata

SEQ ID NO: 26 - DNA, 22 bp, Artificial
  KRAS codon 61_R2
taaacccacctataatggtgaa

SEQ ID NO: 27 - DNA, 26 bp, Artificial
  KRAS codon 12, 13_F
ggtgagtttgtattaaaaggtactgg

SEQ ID NO: 28 - DNA, 20 bp, Artificial
  KRAS codon 12, 13_R
tcctgcaccagtaatatgca

SEQ ID NO: 29 - DNA, 20 bp, Artificial
  STK11 exon1_F
ccgttggcacccgtgaccta

SEQ ID NO: 30 - DNA, 20 bp, Artificial
  STK11 exon1_R
accatcagcaccgtgactgg

SEQ ID NO: 31 - DNA, 18 bp, Artificial
  STK11 exon2_F
gggcggatcacaaggtca

SEQ ID NO: 32 - DNA, 19 bp, Artificial
  STK11 exon2_R
aggagacgggaagaggagc

SEQ ID NO: 33 - DNA, 18 bp, Artificial
  STK11 exon3_F
tgtgcccagagcaagagc

SEQ ID NO: 34 - DNA, 20 bp, Artificial
  STK11 exon3_R
gcagaagaatggcgtgaacc

SEQ ID NO: 35 - DNA, 19 bp, Artificial
  STK11  exon4&5_F
aggagacgggaagaggagc

SEQ ID NO: 36 - DNA, 20 bp, Artificial
  STK11  exon4&5_R
tgaaccaccatctgccgtat

SEQ ID NO: 37 - DNA, 20 bp, Artificial
  STK11 exon6_F
tgactgaccacgcctttctt

SEQ ID NO: 38 - DNA, 18 bp, Artificial
  STK11 exon6_R
tgagggacctggcaaacc

SEQ ID NO: 39 - DNA, 21 bp, Artificial
  STK11 exon7_F
cagggtctgtcagggttgtcc

SEQ ID NO: 40 - DNA, 19 bp, Artificial
  STK11 exon7_R
ccgtccgctgctctgtctt

SEQ ID NO: 41 - DNA, 19 bp, Artificial
  STK11 exon8_F
actgcttctgggcgtttgc

SEQ ID NO: 42 - DNA, 18 bp, Artificial
  STK11 exon8_R
aggtgggctggaggcttt

SEQ ID NO: 43 - DNA, 20 bp, Artificial
  STK11 exon9_F
ggttctgtgctggcatttcg

SEQ ID NO: 44 - DNA, 19 bp, Artificial
  STK11 exon9_R
ggctctgacgctggtggat

SEQ ID NO: 45 - DNA, 19 bp, Artificial
  STK11 exon10a_F
tgcccaggctgacctcttc

SEQ ID NO: 46 - DNA, 21 bp, Artificial
  STK11 exon10a_R
cgatggcgtttctcgtgtttt

SEQ ID NO: 47 - DNA, 22 bp, Artificial
  STK11 exon10b_F
ggatttgagctgtggctgtgag

SEQ ID NO: 48 - DNA, 20 bp, Artificial
  STK11 exon10b_R
aacaccgtgactgccgacct

SEQ ID NO: 49 - DNA, 37 bp, Artificial
  TP53 Exon 2,3,4_F
tgtaaaacgacggccagtgccgagctgtctcagacac

SEQ ID NO: 50 - DNA, 36 bp, Artificial
  TP53 Exon 2,3,4_R
caggaaacagctatgaccgaaatgcagggggatacg

SEQ ID NO: 51 - DNA, 37 bp, Artificial
  TP53 Exon 2,3_F
tgtaaaacgacggccagtggagtgcttgggttgtggt

SEQ ID NO: 52 - DNA, 35 bp, Artificial
  TP53 Exon 2,3_R
caggaaacagctatgacccggcaagggggactgta

SEQ ID NO: 53 - DNA, 36 bp, Artificial
  TP53 Exon 4_F
tgtaaaacgacggccagtgacttcctgaaaacaacg

SEQ ID NO: 54 - DNA, 38 bp, Artificial
  TP53 Exon 4_R
caggaaacagctatgacccacacattaagtgggtaaac

SEQ ID NO: 55 - DNA, 37 bp, Artificial
  TP53 Exon 5,6_F
tgtaaaacgacggccagttttctttgctgccgtcttc

SEQ ID NO: 56 - DNA, 38 bp, Artificial
  TP53 Exon 5,6_R
caggaaacagctatgaccttgcacatctcatggggtta

SEQ ID NO: 57 - DNA, 36 bp, Artificial
  TP53 Exon 7_F
tgtaaaacgacggccagtgaccatcctggctaacgg

SEQ ID NO: 58 - DNA, 39 bp, Artificial
  TP53 Exon 7_R
caggaaacagctatgacccacaggttaagaggtcccaaa

SEQ ID NO: 59 - DNA, 38 bp, Artificial
  TP53 Exon 8,9_F
tgtaaaacgacggccagttttgggacctcttaacctgt

SEQ ID NO: 60 - DNA, 39 bp, Artificial
  TP53 Exon 8,9_R
caggaaacagctatgacccaggcaaagtcatagaaccat

SEQ ID NO: 61 - DNA, 38 bp, Artificial
  TP53 Exon 10_F
tgtaaaacgacggccagtcatgttgcttttgtaccgtc

SEQ ID NO: 62 - DNA, 39 bp, Artificial
  TP53 Exon 10_R
caggaaacagctatgaccggcaagaatgtggttatagga

SEQ ID NO: 63 - DNA, 37 bp, Artificial
  TP53 Exon 11_F
tgtaaaacgacggccagtaagggaagattacgagact

SEQ ID NO: 64 - DNA, 38 bp, Artificial
  TP53 Exon 11_R
caggaaacagctatgacctaagctggtatgtcctactc

SEQ ID NO: 65 - DNA, 20 bp, Artificial
  TP53_cDNA_1_F
tcgggctgggagcgtgcttt

SEQ ID NO: 66 - DNA, 20 bp, Artificial
  TP53_cDNA_1_R
agctgcacagggcaggtctt

SEQ ID NO: 67 - DNA, 20 bp, Artificial
  TP53_cDNA_2_F
ggacagccaagtctgtgact

SEQ ID NO: 68 - DNA, 20 bp, Artificial
  TP53_cDNA_2_R
ggagaggagctggtgttgtt

SEQ ID NO: 69 - DNA, 20 bp, Artificial
  TP53_cDNA_3_F
ccatcctcaccatcatcaca

SEQ ID NO: 70 - DNA, 21 bp, Artificial
  TP53_cDNA_3_R
gctgtcagtggggaacaagaa

(19) United States
(12) Patent Application Publication
(10) Pub. No.: US 2022/0356533 A1
(43) Pub. Date: Nov. 10, 2022

(54) BIOMARKER COMPOSITION FOR DIAGNOSING OR PREDICTING PROGNOSIS OF THYROID CANCER, COMPRISING PREPARATION CAPABLE OF DETECTING MUTATION IN PLEKHS1 GENE, AND USE THEREOF

(71) Applicant: THE CATHOLIC UNIVERSITY OF KOREA INDUSTRY-ACADEMIC COOPERATION FOUNDATION, Seoul (KR)

(72) Inventors: Chan Kwon JUNG, Seoul (KR); Seung Hyun JUNG, Daegu (KR); Yeun Jun CHUNG, Yongin-si (KR)

(73) Assignee: THE CATHOLIC UNIVERSITY OF KOREA INDUSTRY-ACADEMIC COOPERATION FOUNDATION, Seoul (KR)

(21) Appl. No.: 17/774,424

(22) PCT Filed: Oct. 23, 2020
(86) PCT No.: PCT/KR2020/014570

§ 371 Date: May 4, 2022

(30) Foreign Application Priority Data
Nov. 8, 2019 (KR) 10-2019-0142180

(51) Int. Cl. C12Q 1/6886; C12Q 1/6827

(52) U.S. Cl. CPC C12Q 1/6886; C12Q 1/6827; C12Q 2600/118

(57) ABSTRACT

The present invention relates to: a biomarker composition for diagnosing or predicting the prognosis of thyroid cancer, comprising a preparation capable of detecting a mutation in a PLEKHS1 promoter gene; and a use thereof. The biomarker composition for diagnosing or predicting the prognosis of thyroid cancer of the present invention confirms whether a mutation is present in a PLEKHS1 promoter gene, and thus can provide information needed for diagnosing metastatic (distant metastatic) differentiated thyroid cancer, and also confirms whether a mutation is present in BRAF, TERT promoter, three types of RAS and a TP53 gene in addition to the PLEKHS1 promoter gene, and thus, with respect to radioactive iodine therapy response and survival, can classify the prognosis of a metastatic differentiated thyroid cancer patient into one of three prognosis groups, and predict the same.

### FIELD OF THE DISCLOSURE

The present invention relates to a biomarker composition for diagnosing or predicting the prognosis of thyroid cancer, comprising a preparation capable of detecting a mutation in a PLEKHS1 promoter gene, and a use thereof.

### DESCRIPTION OF RELATED ART

Differentiated thyroid cancer originates from follicular cells and accounts for 95% or more of all thyroid cancers. Differentiated thyroid cancer is histologically divided into papillary thyroid carcinoma (PTC), follicular thyroid carcinoma (FTC) and poorly differentiated thyroid carcinoma (PDTC). Although most differentiated thyroid cancer shows a very good prognosis compared to other types of cancer, 1 to 9% of all patients also have distant metastasis at the time when the cancer is found, and distant metastasis occurs in 7 to 23% during follow-up observation after initial treatment. Distant metastasis is the leading cause of death from thyroid cancer, and patients with distant metastasis have 65% and 75% chances of dying from thyroid cancer in 5 and 10 years, respectively. Known factors of distant metastasis include lateral neck lymph node metastasis, specific histological types (papillary thyroid carcinoma of tall cell variant, columnar cell variant and hobnail variant, vascular invasive follicular carcinoma, widespread invasive follicular carcinoma or poorly differentiated carcinoma), a mutation in the TERT promoter or multiple occurring mutations, and the like. However, such pathological and genetic factors alone cannot accurately predict the risk of distant metastasis and cannot determine the prognosis of individual patients.

The most common cancer-inducing gene mutations in differentiated thyroid cancer are somatic mutations of BRAF and RAS genes. The progression of differentiated thyroid cancer to a worse type of cancer is highly associated with the accumulation of cancer-inducing gene mutations such as TP53, PIK3CA, AKT1, TERT, a histone methyltransferase gene, a mismatch repair gene or a chromatin remodeling complex. Mutations occurring in the promoter region of a gene may regulate the transcriptional ability of the gene by altering or interfering with the binding of transcription factors to cis-acting DNA-sequences. For example, a mutation in the TERT promoter activates telomerase by increasing the promoter activity and transcriptional ability of a TERT gene, occurs in 10 to 20% of cases of differentiated thyroid cancer, and is highly associated with increases in distant metastasis, recurrence and mortality. Recently, new mutations that regulate gene transcription by whole genome sequencing have been sought after, and as an example, mutations in the transcriptional regulatory regions of PLEKHS1, WDR74 and SDHD genes have been reported to affect cancer development. These gene mutations are likely to be utilized as markers to predict clinical outcomes such as a treatment response assessment in patients with differentiated thyroid cancer. However, since studies on the gene mutation have not yet been performed, genetic test results can only partially predict the clinical outcomes of patients with differentiated thyroid cancer, despite the molecular pathological development of thyroid cancer. Therefore, there still remains an unmet demand for the development of a genetic marker for predicting the prognosis of patients with thyroid cancer.

Korean Patent Application No. 10-2015-0010141 relates to a method for diagnosing the metastasis of primary thyroid cancer and a diagnosis kit using the same, and provides Slit2 as a new biomarker and a new molecular therapy target based on the fact that deterioration in activity of the Slit-Robo pathway is associated with a poor prognosis of thyroid cancer.

However, no studies or descriptions have been disclosed on the correlation between the PLEKHS1 gene and the diagnosis or prognosis of thyroid cancer, particularly differentiated thyroid cancer.

### SUMMARY

As a result of intensive studies to provide a biomarker capable of diagnosing or predicting the prognosis of thyroid cancer, the present inventors confirmed that when a mutation occurred in a PLEKHS1 gene, differentiated thyroid cancer with distant metastasis could be diagnosed and the prognosis of the patient could be predicted to be poor, thereby completing the present invention.

Therefore, the present invention can provide a biomarker composition for diagnosing or predicting the prognosis of thyroid cancer, comprising a preparation capable of detecting a mutation in a PLEKHS1 promoter gene; and a use thereof.

The present invention provides a biomarker composition for diagnosing or predicting the prognosis of thyroid cancer, comprising a preparation capable of detecting a mutation in a PLEKHS1 promoter gene, and a kit for diagnosing or predicting the prognosis of thyroid cancer comprising the same.

According to a preferred embodiment of the present invention, the composition may further comprise a preparation capable of detecting a mutation in any one or more genes selected from the group consisting of a TERT promoter gene, a TP53 gene, an STK11 gene, a BRAF gene and an RAS gene.

According to a preferred embodiment of the present invention, the prognosis may be likely to be resistance to radioactive iodine therapy or death.

According to a preferred embodiment of the present invention, the diagnosis may be differentiated thyroid cancer with distant metastasis.

According to a preferred embodiment of the present invention, the thyroid cancer may be any one or more selected from the group consisting of a papillary thyroid carcinoma (PTC), a follicular thyroid carcinoma (FTC) and a poorly differentiated thyroid carcinoma (PDTC).

According to a preferred embodiment of the present invention, the composition may further comprise a preparation capable of confirming the copy number variation of the loss of the long arm (q) of chromosome 22.

According to a preferred embodiment of the present invention, the loss of the long arm (q) of chromosome 22 may be a loss of q11.1-q13.33 regions of chromosome 22.

According to a preferred embodiment of the present invention, the composition may further comprise a preparation capable of confirming the copy number variations of any one or more selected from the group consisting of

a) gain of the long arm (q) of chromosome 1;

b) loss of the short arm (p) of chromosome 9;

c) loss of the long arm (q) of chromosome 9; and

d) loss of the long arm (q) of chromosome 11.

According to a preferred embodiment of the present invention,

a) the gain of the long arm (q) of chromosome 1 may be a gain of q12-q44 regions of chromosome 1;

b) the loss of the short arm (p) of chromosome 9 may be a loss of p24.3-p13.1 and p13.1-p11.2 regions of chromosome 9;

c) the loss of the long arm (q) of chromosome 9 may be a loss of q12-q31.1 and q31.1-q34.3 regions of chromosome 9; and

d) the loss of the long arm (q) of chromosome 11 may be a loss of q11.2-q22.1, q22.1-q23.2 and q23.2-q24.3 regions of chromosome 11.

The present invention also provides a method for providing information for diagnosing or predicting the prognosis of thyroid cancer, the method including: a) obtaining a gene from a sample isolated from an individual;

b) confirming whether a mutation is present in any one or more genes selected from the group consisting of a PLEKHS1 promoter gene, a TERT promoter gene and a TP53 gene in the gene of Step a); and

c) confirming whether a mutation is present in any one or more genes selected from the group consisting of a BRAF gene and an RAS gene when no mutation is found in Step b).

According to a preferred embodiment of the present invention, the sample in Step a) may be any one or more selected from the group consisting of tumor, blood, urine and saliva.

According to a preferred embodiment of the present invention, when the mutation in Step b) is confirmed, the individual may be determined to be likely to exhibit resistance to radioactive iodine therapy and to die.

According to a preferred embodiment of the present invention, when the mutation in Step c) occurs, the individual may be determined to exhibit resistance to radioactive iodine therapy, and

when the mutation is not found, the individual may show a good prognosis.

According to a preferred embodiment of the present invention, in Step b), the copy number variations of any one or more selected from the group consisting of,
among the genes isolated in Step a),

a) gain of the long arm (q) of chromosome 1;
b) loss of the long arm (q) of chromosome 9;
c) loss of the long arm (q) of chromosome 11; and
d) loss of the long arm (q) of chromosome 22 may be further confirmed.

According to a preferred embodiment of the present invention, when the copy number variation is confirmed, a patient with differentiated thyroid cancer may be determined to be likely to exhibit resistance to radioactive iodine therapy and to die.

As used herein, the term ‘diagnosis’ refers to the determination of an actual condition of the disease of a patient in all aspects in a broad sense. The content of the determination is the disease entity, the pathogenesis, the severity, the detailed aspect of a disease, the presence or absence of complications, and the like. In the present invention, the diagnosis preferably includes determining distant metastasis thyroid cancer or the risk of developing the same.

As used herein, ‘prognosis’ refers to a prospect or preliminary assessment of the medical outcome of a disease, and means predicting, for example, a bad or good outcome (for example, possibility of long-term survival). A negative (poor) prognosis or bad outcome includes prediction of recurrence, disease progression (for example, cancer growth or metastasis, or resistance to drugs or treatment), or possibility of death, and a positive (good) prognosis or good outcome includes prediction of the disease being cured (for example, a disease-free state), alleviation (for example, cancer eradication) or stabilization. Prediction of prognosis (or diagnosis of prognosis) may suggest clues for future treatment of thyroid cancer, including particularly whether patients with thyroid cancer will be subjected to radioactive iodine therapy. Prognosis prediction also includes the patient's response to a thyroid cancer therapeutic agent and the prediction of the treatment course.

Patients with differentiated thyroid cancer (metastatic differentiated thyroid cancer) which causes distant metastasis are known to have a low survival rate. Half or more of these patients exhibit resistance to radioactive iodine (RAI) therapy, which contributes to a bad prognosis. It is important to identify a patient with a high risk of distant metastasis in the early stage of the treatment process. Metastatic differentiated thyroid cancer often has a mutation in the TERT promoter, but since half or more of patients with metastatic differentiated thyroid cancer do not have the mutation, the presence of the mutation alone is not sufficient to predict the risk of distant metastasis.

Some cases of differentiated thyroid cancer may exhibit resistance to radioactive iodine therapy at a site of metastasis and may also progress to a poorly differentiated carcinoma or anaplastic cancer. These tumors often carry mutations of various genes including the WNT signaling pathways, PI3K/AKT pathways, SWI/SNF chromatin remodeling complexes, histone methyltransferases, DNA mismatch repairs and tumor suppressors with the TERT promoters. Among the mutations that affect the PI3K/AKT pathway, PIK3CA and AKT1 mutations are associated with tumor progression and distant metastasis of differentiated thyroid cancer. The present invention is the first to present a recurrent mutation in the PLEKHS 1 promoter (13%) as a potential prognostic factor for differentiated thyroid cancer metastasis. The mutation in the PLEKHS 1 promoter was found in 14% (3/21) of metastatic differentiated thyroid cancers without the mutation in the TERT promoter (FIGS. 1A to 1G).

The pleckstrin homology domain containing S1 (PLEKHS1) gene includes a pleckstrin homology domain capable of playing a role of intracellular signaling. The clinical significance of the mutation in the PLEKHS1 promoter in human cancer and its role in tumorigenesis are unknown.

The greatest challenge in managing metastatic differentiated thyroid cancer is to strike a balance between aggressive treatment and active monitoring. In the case of metastatic tumors resistant to radioactive iodine therapy, an additional treatment method needs to be tailored to a patient depending on the degrees of metastatic diseases, symptoms, comorbidities and progression rates. Currently, the American Thyroid Association management guidelines for differentiated thyroid cancer do not recommend molecular testing for the purpose of prognosis of patients with radioactive-iodine-therapy-resistant or metastatic differentiated thyroid cancer due to the lack of strong evidence.

The present inventors developed a genetic discriminator capable of classifying patients with metastatic differentiated thyroid cancer into three prognosis groups (good, intermediate, and poor). Patients in the good prognosis group showed a very slow progressing disease, whereas half or more of the patients in the poor prognosis group died of progressive disease within 10 years. Therefore, this genetic discriminator may be a guide for clinicians to decide whether to give a patient systemic treatment. Further, patients in the poor prognosis group can benefit from treatment by receiving systemic therapy in the early stage.

Thus, the present inventors intended to confirm recurrent somatic mutations and copy number variations in differentiated thyroid cancer by performing targeted deep sequencing capable of analyzing the coding region and non-coding region of 157 major cancer-related genes. In addition, the present inventors intended to develop a genetic discriminator capable of predicting clinical outcomes including therapeutic responses to differentiated thyroid cancer that induced distant metastasis.

Therefore, the present invention can provide a biomarker composition for diagnosing or predicting the prognosis of thyroid cancer, comprising a preparation capable of detecting a mutation in a PLEKHS1 promoter gene.

The mutation in the PLEKHS1 promoter gene means that a mutation occurs on chromosome 10. Specifically, when cysteine (C) at the position of 115,511,590 bp or 115,511,593 bp of chromosome 10 is mutated to threonine (T) (C590T or C593T), it is possible to utilize the mutation for diagnosing or predicting the prognosis of thyroid cancer.

According to a preferred embodiment of the present invention, the composition may further comprise a preparation capable of detecting a mutation in any one or more genes selected from the group consisting of a TERT promoter gene, a TP53 gene, an STK11 gene, a BRAF gene and an RAS gene.

The mutation in the TERT promoter gene means that a mutation occurs on chromosome 5. Specifically, when cysteine (C) at the position of 1,295,228 bp or 1,295,250 bp as a promoter hotspot site of the TERT gene of chromosome 5 is mutated to threonine (T)

(C228T or C250T), it is possible to utilize the mutation for diagnosing or predicting the prognosis of thyroid cancer.

It is possible to utilize the mutation of the TP53 gene for diagnosing or predicting the prognosis of thyroid cancer when methionine (M) at position 237 of the short arm (p) is mutated to isoleucine (I) (p.M237I), tyrosine (Y) at position 220 of the short arm is mutated to cysteine (C) (p.Y220C), or a frame shift (fs) mutation (p.G117fs) of glycine (G) of the short arm occurs. However, since the TP53 gene produces a p53 protein consisting of 393 amino acids as a tumor suppressor gene, and most mutations in TP53 occur in a gene region encoding a very wide site from position 102 to position 292 of amino acids, the mutation is not limited to the specific position of the mutation revealed in the present invention.

It is possible to utilize the mutation of the STK11 gene for diagnosing or predicting the prognosis of thyroid cancer when phenylalanine (F) at position 354 of the short arm (p) is mutated to leucine (L) (p.F354L).

It is possible to utilize the mutation of the BRAF gene for diagnosing or predicting the prognosis of thyroid cancer when valine (V) at position 600 of the short arm (p) is mutated to glutamic acid (E) (p.V600E).

The mutation of the RAS gene may occur at hotspot codon 61, and specifically, it is possible to utilize the mutation for diagnosing or predicting the prognosis of thyroid cancer when glutamine (Q) at codon 61 position in the NRAS, HRAS or KRAS gene is mutated to arginine (R) (p.Q61R), leucine (L) (p.Q61L), or lysine (K) (p.Q61K), or when glutamine (Q) at codon 12 or 13 position is mutated to arginine (R) (p.Q12R, p.Q13R), alanine (A) (p.Q12A, p.Q13A), cysteine (C) (p.Q12C, p.Q13C), aspartic acid (D) (p.Q12D, p.Q13D), serine (S) (p.Q12S, p.Q13S), or valine (V) (p.Q12V, p.Q13V).

The preparation capable of detecting a mutation is a preparation needed for detecting the mutation by amplifying a mutated gene site, and the concept includes all preparations that can be used for gene amplification at the level of those skilled in the art. Preferably, the preparation may be a preparation needed for a polymerase chain reaction (PCR), and the PCR may be a quantitative PCR (qPCR), a real-time PCR, a reverse transcription PCR (RT-PCR), a solid phase PCR, a competitive PCR, an overlap-extension PCR, a multiplex PCR, a nested PCR, an inverse PCR, a ligation-mediated PCR, an intersequence-specific PCR (ISSR), a methylation-specific PCR (MSP), a colony PCR, a miniprimer PCR, a nanoparticle-assisted PCR (nanoPCR), a thermal asymmetric interlaced PCR (TAIL-PCR), a touchdown (step-down) PCR, a hot start PCR, an in silico PCR, an allele-specific PCR, an assembly PCR, an asymmetric PCR, a dial-out PCR, a digital PCR (dPCR) or helicase-dependent amplification.

According to a preferred embodiment of the present invention, the prognosis may be likely to be resistance to radioactive iodine therapy or death.

The resistance to radioactive iodine therapy means that an individual exhibits a lower effect when subjected to radioactive iodine therapy than a normal group. Specifically, during radioactive iodine therapy, in a case corresponding to any one of 1) when at least one metastatic lesion does not take up radioactive iodine; 2) when the size of a lesion continues to increase even though the lesion takes up radioactive iodine; 3) when the size of a distant metastatic lesion increases one or more years after radioactive iodine therapy; or 4) when there is a lesion that continues even after the cumulative dose of radioactive iodine therapy is 600 mCi or higher, it can be seen to exhibit resistance to radioactive iodine therapy (Example <1-2>).

To be likely to die means that there is a possibility of dying of thyroid cancer within 10 years (follow-up observation period) after a patient is diagnosed with distant metastasis of thyroid cancer (Example <1-3>).

According to a preferred embodiment of the present invention, the diagnosis may be differentiated thyroid cancer with distant metastasis.

According to a preferred embodiment of the present invention, the thyroid cancer may be differentiated thyroid cancer, more preferably, any one or more selected from the group consisting of papillary thyroid carcinoma (PTC), follicular thyroid carcinoma (FTC) and poorly differentiated thyroid carcinoma (PDTC).

According to a preferred embodiment of the present invention, the composition may further include a preparation capable of confirming the copy number variation of the loss of the long arm (q) of chromosome 22, and the loss of the long arm (q) of chromosome 22 may be a loss of q11.1-q13.33 regions of chromosome 22.

The chromosome may be a human chromosome.

The copy number variation is a DNA fragment showing a change in copy number compared to a reference genome, and is one of the structural variations of a gene in which a specific base sequence of 1 kb or more is deleted (0 n or 1 n) or acquired (3 n or more).

The preparation capable of confirming the copy number variation may include all preparations needed for confirming the copy number variation at the level of those skilled in the art.

When the short arm or long arm of a chromosome is lost, the length of the chromosome may be shortened. That is, when a mutation in which the short arm or long arm of the chromosome is lost occurs, the length becomes shorter than that of the wild type, and thus the presence or absence of the mutation can be confirmed by confirming the length of the chromosome.

When the short arm or long arm of a chromosome is gained, the length of the chromosome may be lengthened. That is, when a mutation in which the short arm or long arm of the chromosome is gained occurs, the length becomes longer than that of the wild type, and thus the presence or absence of the mutation can be confirmed by confirming the length of the chromosome.

According to a preferred embodiment of the present invention, the composition may further comprise a preparation capable of confirming the copy number variations of any one or more selected from the group consisting of

a) gain of the long arm (q) of chromosome 1;

b) loss of the short arm (p) of chromosome 9;

c) loss of the long arm (q) of chromosome 9; and

d) loss of the long arm (q) of chromosome 11.

According to a preferred embodiment of the present invention, a) the gain of the long arm (q) of chromosome 1 may be a gain of q12-q44 regions of chromosome 1;

b) the loss of the short arm (p) of chromosome 9 may be a loss of p24.3-p13.1 and p13.1-p11.2 regions of chromosome 9;

c) the loss of the long arm (q) of chromosome 9 may be a loss of q12-q31.1 and q31.1-q34.3 regions of chromosome 9; and

d) the loss of the long arm (q) of chromosome 11 may be a loss of q11.2-q22.1, q22.1-q23.2 and q23.2-q24.3 regions of chromosome 11.

The present invention may also provide a kit for diagnosing or predicting the prognosis of thyroid cancer, comprising the biomarker composition. The kit may include a primer or probe set capable of detecting a gene in which a mutation may occur.

The present invention also provides a method for providing information for diagnosing or predicting the prognosis of thyroid cancer, comprising: a) obtaining a gene from a sample isolated from an individual;

b) confirming whether a mutation is present in any one or more genes selected from the group consisting of a PLEKHS1 promoter gene, a TERT promoter gene and a TP53 gene in the gene of Step a); and

c) confirming whether a mutation is present in any one or more genes selected from the group consisting of a BRAF gene and an RAS gene when no mutation is found in Step b).

The concept of an individual includes all mammals including humans, but may preferably refer to a human, more preferably a patient with thyroid cancer.

According to a preferred embodiment of the present invention, the sample in Step a) may be any one or more selected from the group consisting of tumor, blood, urine and saliva, and may preferably be a tumor.

The gene may be DNA.

According to a preferred embodiment of the present invention, when the mutation in Step b) is confirmed, the individual may be determined to be likely to exhibit resistance to radioactive iodine therapy and to die.

Since the mutation in the PLEKHS1 promoter gene, TERT promoter gene or TP53 gene and the possibility of resistance to radioactive iodine therapy or death are the same as the concept used in the biomarker composition, the above description can be referred to for an explanation thereof.

According to a preferred embodiment of the present invention, when the mutation in Step c) occurs, the individual may be determined to exhibit resistance to radioactive iodine therapy, and

when the mutation is not found, the individual may be determined to show a good prognosis.

Since the resistance to radioactive iodine therapy is the same as the concept used in the biomarker composition, the above description can be referred to for an explanation thereof.

The good prognosis means that a patient with thyroid cancer can survive without showing resistance to radioactive iodine therapy.

According to a preferred embodiment of the present invention, in Step b), the copy number variations of any one or more selected from the group consisting of,
among the genes isolated in Step a),
a) gain of the long arm (q) of chromosome 1;
b) loss of the long arm (q) of chromosome 9;
c) loss of the long arm (q) of chromosome 11; and
d) loss of the long arm (q) of chromosome 22 may be further confirmed.

Since the gain or loss or copy number variation of the chromosome is the same as the concept used in the biomarker, the above description can be referred to for an explanation thereof.

According to a preferred embodiment of the present invention, when the copy number variation is confirmed, a patient with differentiated thyroid cancer may be likely to exhibit resistance to radioactive iodine therapy and to die.

Since the copy number variation and the possibility of resistance to radioactive iodine therapy or death is the same as the concept used in the biomarker, the above description can be referred to for an explanation thereof.

The biomarker composition for diagnosing or predicting the prognosis of thyroid cancer of the present invention confirms whether a mutation is present in a PLEKHS1 promoter gene, and thus can provide information needed for diagnosing metastatic (distant metastatic) differentiated thyroid cancer, and also confirms whether a mutation is present in BRAF, TERT promoter, three types of RAS and a TP53 gene in addition to the PLEKHS1 promoter gene, and thus, with respect to radioactive iodine therapy response and survival, can classify the prognosis of a metastatic differentiated thyroid cancer patient into one of three prognosis groups, and predict the same.

### BRIEF DESCRIPTION OF THE DRAWINGS

FIGS. 1A to 1D illustrate the somatic mutations and copy number variations that are repeatedly found in differentiated thyroid cancer. An analysis of clinicopathological characteristics and gene mutations of a total of 61 patients was performed using 46 primary tumor tissues and 15 distant metastatic tumor tissues.

FIGS. 1E and 1F illustrate the somatic mutations and copy number variations that are repeatedly found in differentiated thyroid cancer. An analysis of clinicopathological characteristics and gene mutations of a total of 61 patients was performed using 38 primary tumor tissues.

FIG. 1G illustrates the somatic mutations and copy number variations that are repeatedly found in differentiated thyroid cancer. An analysis of clinicopathological characteristics and gene mutations of a total of 61 patients was performed using 6 primary and distant metastatic tumor tissues.

FIG. 2 illustrates the mutation frequencies of the TERT promoter, BRAF, and the PLEKHS1 promoter found in a total of 61 patients with differentiated thyroid cancer accompanied by distant metastasis.

FIG. 3 illustrates the results of analyzing variant allele frequencies of mutations against PLEKHS1, TERT, BRAF, RAS and STK11.

FIG. 4 illustrates copy number variations in 51 differentiated thyroid cancers with distant metastases stratified by cancer type.

FIGS. 5A and 5B illustrate repetitive 22q11.1-q13.33 gene copy number loss in differentiated thyroid cancer accompanied by distant metastasis.

FIGS. 6A and 6B illustrate the results of analyzing cancer-specific survival rates according to bone metastasis (A), the presence or absence of high-risk gene mutations (mutations in TERT, PLEKHS1 or TP53)(B), and 11 q chromosome loss in 61 patients with differentiated thyroid cancer with distant metastasis. A detailed analysis of 38 patients with papillary thyroid carcinoma also showed similar results (D to F).

FIG. 7 illustrates the risk stratification for radioactive iodine therapy response and cancer-specific survival rate according to mutation patterns for BRAF, the three types of RAS, the TERT promoter, the PLEKHS1 promoter, and the TP53 gene. Patients without such gene mutations are a group with the best prognosis. When any one of the TERT promoter, the PLEKHS1 promoter, and the TP53 gene is mutated, it is a group with the worst prognosis, and when the above three genes are not mutated, but the BRAF or RAS gene is mutated, it is a group showing an intermediate prognosis.

FIGS. 8A and 8B illustrate the types of gene mutations in 84 patients with poorly differentiated thyroid carcinoma and 33 patients with anaplastic carcinoma using cBioPortal for Cancer Genomics (http://cbioportal.org) after the types were re-analyzed in a thyroid cancer study conducted at the Memorial Sloan Kettering Cancer Center. The mutation in the TERT promoter and the mutation in TP53 were found in 50% and 25%, respectively. PIK3CA mutation was found in 7%. Of the eight thyroid cancers with PIK3CA mutation, seven had the mutation in the TERT promoter at the same time, and two had mutations in the TERT promoter and the TP53 gene at the same time. No AKT1 mutation was found. Mutation rates for other genes were only 0% to 7%, which were also mostly found together with the TERT promoter or mutations in TP53.

### DETAILED DESCRIPTION OF THE INVENTION

### EXAMPLE 1

Characteristics of Patients Studied

<1-1> Patients and Tissue Samples to be Studied

61 patients with differentiated thyroid cancer treated at Seoul St. Mary's Hospital between 2006 and 2017 included 48 patients with papillary thyroid carcinomas, 9 patients with follicular thyroid carcinomas, and 4 patients with poorly differentiated thyroid carcinomas. This study was approved and conducted by the Institutional Review Board of the Catholic University of Korea, Seoul St. Mary's Hospital. All pathological slides were classified by a pathologist specializing in endocrine pathology in accordance with the latest diagnostic criteria of the World Health Organization as of 2017. Of the 61 patients with differentiated thyroid cancer included in the study, 20 had already been found to have distant metastasis at the time of diagnosis, and the remaining 41 had distant metastasis during a follow-up observation period after thyroid surgery. Distant metastasis for the 32 patients was confirmed in surgical specimens, and the remaining 29 could not be histologically examined, and thus were diagnosed by imaging images such as whole-body scans, computed tomography, magnetic resonance imaging images or positron emission tomography. To validate the clinical usefulness of a distant-metastasis-specific gene, 75 patients with papillary thyroid carcinomas who had no distant metastasis at the time of diagnosis were additionally included. The cancer stage was classified based on the 8th Edition of the American Joint Committee on Cancer. Cancer stage and treatment policy were determined by a multidisciplinary cooperative medical team consisting of an internal medicine department, a department of surgery, a department of imaging medicine, a department of nuclear medicine, and a department of pathology.

<1-2> Evaluation of Response to Radioactive Iodine Therapy

All 61 patients with distant metastasis received radioactive iodine therapy. Responsiveness to radioactive iodine therapy was evaluated by comprehensively analyzing whole body iodine 131 scans, serum thyroglobulin values, or imaging images such as computed tomography, magnetic resonance imaging images or positron emission tomography. Radioactive iodine therapy results were divided into responsive and non-responsive (resistance or tolerance). A case of exhibiting resistance to radioactive iodine therapy was defined as any one of the following: 1) when at least one metastatic lesion did not take up radioactive iodine, 2) when the size of a lesion continued to increase even though the lesion took up radioactive iodine, 3) when the size of a distant metastatic lesion increased one or more years after radioactive iodine therapy, or 4) when there was a lesion that remained even after the cumulative dose of radioactive iodine therapy was 600 mCi or higher.

<1-3> General Characteristics of Patients with Differentiated Thyroid Cancer Accompanied by Distant Metastasis

The general characteristics of patients with differentiated thyroid cancer accompanied by distant metastasis analyzed according to Examples <1-1>and <1-2>are shown in [Table 1].

PTC means papillary thyroid carcinoma; FTC means follicular thyroid carcinoma; PDTC means poorly differentiated thyroid carcinoma; and RAI means radioactive iodine.

TABLE 1

| | | | | | TERT | | PLEKHS1 | | | |
|---|---|---|---|---|---|---|---|---|---|---|
| | BRAF | | RAS | | promoter | | promoter | | TP53 | |
| | mutation | p- | mutation | p- | mutation | p- | mutation | | mutation | p- |
| Features | (n = 31) | value | (n = 13) | value | (n = 28) | value | (n = 6) | p-value | (n = 2) | value |
| Distant | | | | | | | | | | |
| metastasis | | | | | | | | | | |
| occurrence | | | | | | | | | | |
| age (median) | | | | | | | | | | |
| Distant | 66 | 0.010 | 59 | 1.000 | 65 | 0.001 | 67 | 0.2784 | 72 | 0.1277 |
| metastasis | | | | | | | | | | |
| occurrence | | | | | | | | | | |
| age (median) | | | | | | | | | | |
| Age group | | 0.031 | | 0.715 | | 0.002 | | 1.000 | | 1.000 |
| <45 years old | 3 (23%) | | 2 (15%) | | 1 (8%) | | 1 (8%) | | 0 | |
| (n = 13) | | | | | | | | | | |
| ≥45 years old | 28 (58%) | | 11 (22%) | | 27 (56%) | | 5 (10%) | | 2 (4%) | |
| (n = 48) | | | | | | | | | | |
| Sex | | 0.849 | | 0.520 | | 0.340 | | 0.655 | | 0.093 |
| Female (n = 42) | 21 (50%) | | 8 (19%) | | 21 (50%) | | 5 (12%) | | 0 | |
| Male (n = 19) | 10 (53%) | | 5 (27%) | | 7 (37%) | | 1 (5%) | | 2 (11%) | |
| Diagnosis | | <0.001 | | <0.001 | | 0.449 | | 0.406 | | 0.009 |
| PTC (n = 48) | 31 (65%) | | 2 (4%) | | 24 (50%) | | 6 (13%) | | 0 | |
| FTC (n = 9) | 0 | | 8 (89%) | | 3 (33%) | | 0 | | 1 (11%) | |
| PDTC (n = 4) | 0 | | 3 (75%) | | 1 (25%) | | 0 | | 1 (25%) | |
| Histological | | 0.674 | | 0.013 | | 0.117 | | 0.200 | | 0.151 |
| type | | | | | | | | | | |
| Non-aggressive | 18 (49%) | | 4 (11%) | | 14 (38%) | | 2 (5%) | | 0 | |
| (n = 37) | | | | | | | | | | |
| Aggressive | 13 (54%) | | 9 (38%) | | 14 (58%) | | 4 (17%) | | 2 (8%) | |
| (n = 24) | | | | | | | | | | |
| Distant | | | | | | | | | | |
| metastasis | | | | | | | | | | |
| Lungs (n = 57) | 29 (50%) | 1.000 | 12 (21%) | 1.000 | 27 (47%) | 0.618 | 5 (9%) | 0.346 | 2 (4%) | 1.000 |
| Bones (n = 20) | 8 (40%) | 0.238 | 10 (50%) | <0.001 | 11 (61%) | 0.562 | 2 (10%) | 1.000 | 2 (10%) | 0.104 |
| Other (n = 8) | 4 (50%) | 1.000 | 3 (38%) | 0.350 | 2 (25%) | 0.269 | 2 (25%) | 0.173 | 1 (13%) | 0.247 |
| RAI | | 0.500 | | 0.288 | | 0.066 | | 0.003 | | 0.164 |
| Effective | 17 (47%) | | 6 (17%) | | 13 (36%) | | 0 | | 0 | |
| (n = 36) | | | | | | | | | | |
| Resistant | 14 (56%) | | 7 (28%) | | 15 (60%) | | 6 (24%) | | 2 (8%) | |
| (n = 25) | | | | | | | | | | |
| Survival state | | 0.425 | | 0.634 | | 0.231 | | 0.136 | | 0.218 |
| Survived | 26 (48%) | | 11 (20%) | | 23 (43%) | | 4 (7%) | | 1 (2%) | |
| (n = 54) | | | | | | | | | | |
| Dead (n = 7) | 5 (71%) | | 2 (29%) | | 5 (71%) | | 2 (29%) | | 1 (14%) | |

Of the 61 patients with differentiated thyroid cancer accompanied by distant metastasis, 48 had papillary thyroid carcinomas, and according to the specific histological type, 21 showed typical papillary thyroid carcinomas, 11 showed a tall cell variant, 5 showed a type showing tall cells only in some parts, 3 showed an invasive follicular variant, 2 showed a columnar cell variant, 2 showed a diffuse sclerosing variant, 2 showed an invasive encapsulated follicular variant, 1 showed a solid variant, and 1 showed a hobnail variant. 9 had follicular thyroid carcinomas and the remaining 4 had poorly differentiated thyroid carcinomas. The organ with the most distant metastasis was the lungs, followed by the bones. 16 (26%) had lung and bone metastases at the same time. 24 showed aggressive histological types (11 with papillary thyroid carcinomas of tall cell variant, 2 with columnar cell variant, 1 with hobnail variant, 6 with extended vascular infiltration encapsulated follicular thyroid carcinomas, and 4 with poorly differentiated thyroid carcinomas). A median of a cumulative radioactive iodine therapy dose was 300 mCi (range: 100 to 900 mCi). 25 (41%) of the 61 patients exhibited resistance to radioactive iodine therapy. After a median of 4.0 (range 0.6 to 11.9 years) during the follow-up observation period, 7 patients died of advanced cancer and the remaining 54 survived or were lost to follow-up within the observation period.

### EXAMPLE 2

Gene Mutation Profile Analysis of Patients

<2-1> DNA Isolation

Genomic DNA was extracted from a 10 μm thick formalin-fixed paraffin-embedded tissue section by utilizing the RecoverAll™ Total Nucleic Acid Isolation Kit (Life Technologies, Carlsbad, Calif., USA). After a tumor tissue compartment was set by utilizing a hematoxylin-eosin stained slide, the position of tumor tissue in an unstained tissue section was confirmed. Only the tumor tissue was accurately excised using a surgical knife while being observed under a microscope. The quantity and quality of extracted DNA was measured using an ND-1000 spectrophotometer (Thermo Fisher Scientific, Waltham, Mass.), and the concentration of DNA was measured using the Qubit 2.0 Fluorometer (Thermo Fisher Scientific) device.

<2-2> Targeted Deep Sequencing

Targeted deep sequencing was performed on 61 tissues of differentiated thyroid cancer by utilizing the OncoChase cancer panel (ConnectaGen, Seoul, Korea). The OncoChase cancer panel includes 157 oncogenes (Table 2). Specifically, 101 paired-end sequence reads were generated using the Illumina HiSeq2000 platform. The sequence reads were matched to a human standard gene (UCSC hg19), and the results were evaluated using the Qualimap. In order to understand the mutation characteristics between primary cancer and metastatic cancer, 6 matching pairs of primary cancer and metastatic cancer were analyzed.

TABLE 2
157 genes

| | Mutation and copy number | Copy number |
|---|---|---|
| Mutant gene | variation | variation |
| NRAS, DDR2, IDH1, ROS1, | MTOR, JAK1, ALK, ERBB4, RAF1, | MYCN, CDK6, MCL1, |
| SMO, GNAQ, RET, HRAS, | CTNNB1, PIK3CA, FGFR3, | CD274, PDCD1LG2, MDM2, |
| MAP2K1, IDH2, GNA11, | PDGFRA, KIT, FGFR4, ESR1, | HS6ST3, RPPH1, NKX2-1, |
| MPL, DNMT3A, XPO1, | EGFR, MET, BRAF, FGFR1, MYC, | CCNE1, MYCL, ACVRL1, |
| NFE2L2, SF3B1, BHL, | JAK2, FGFR2, CCND1, KRAS, | APEX1, ATP11B, BCL2L1, |
| MYD88, RHOA, CSF1R, | ERBB3, CDK4, ATK1, ERBB2, | BCL9, BIRC2, CD44, CCND2, |
| NPM1, EZH2, ABL1, | AMP2K2, JAK3, AR, ARID1A, | CDNK2A1, DCUN1D1, |
| PLEKHS1, WDR74, SDHD, | MDM4, MLH1, KDR, FBXW7, | GAS6, IL6, MYO18A, NKX2- |
| PTPN11, POLE, FLT3, B2M, | TERT, APC, RAC1, CDKN2A, | 8, PNP, PPARG, RPS6KB1, |
| SPOP, SRC, GNAS, U2AF1, | NOTCH1, PTEN, BRCA2, RB1, | TIAF1, ZNF217 |
| MAPK1, MED12, MAP4K3, | TSHR, CDH1, TP53, BRCA1, | |
| MSH2, ZNF2, SHB, HNF1A, | SMAD4, STK11, AURKA, ARAF, | |
| SLC7A8, HSF2BP, CHEK2, | AKT3, BAP1, SOX2, TET2, PIK3R1, | |
| FOXL2, MAX, PPP2R1A, | PDGFRB, RHEB, TSC1, HABP2, | |
| STAT3, PAX5 | WT1, BIRC3, ATM, IGF1R, TSC2, | |
| | NF1, EZH1, NDUFA13, ATK2, | |
| | ASXL1, RUNX1, SMARCB1, NF2, | |
| | APOBEC3B, EIF1AX, KDM6A, | |
| | STAG2, KEAP1, GATA3, RAD51 | |

<2-3> Identification of Somatic Cell Modification and Cancer-Inducing Gene Mutation

Single nucleotide variants (SNVs) were analyzed using MuTect, and insertions-deletions (indels) were analyzed using the Somaticlndel Detector. Somatic mutations located in an exon base sequence were selected using the ANNOVAR package, and functional changes were observed. The following variations were removed for reliable variant calling: 1) read depth within 20 times in tumors, 2) when the frequency of polymorphisms which may be referenced in the 1000 Genomes Project or Exome Aggregation Consortium is 0.1% or more in Asians, and 3) when the minor allele frequency (MAF) is 0.1% or more in the privately owned genetic database of normal Koreans. After variant genes were removed according to the procedure described above, the remaining variations were considered as candidates for somatic mutation, and then variations that overlapped 30 times or more were selected compared to the COSMIC database.

<2-4> Targeted Deep Sequencing Results of Metastatic Thyroid Cancer Genome

The gene mutation profiles of 61 patients with differentiated thyroid cancer accompanied by distant metastasis were analyzed by the targeted deep sequencing of 157 cancer-related genes. An average base sequence depth was 477.1X throughout the genome (range: 104.0X to 1,272.9X) (Table 3).

TABLE 3

| | Sequencing | | Depth range |
|---|---|---|---|
| Sample_ID | read | Mapping read (%) | (average) |
| MTHY01P | 1,234,379 | 1,220,039 (98.8%) | 263.5 |
| MTHY02P | 969,740 | 949,141 (97.9%) | 172.2 |
| MTHY03P | 1,136,436 | 1,110,295 (97.7%) | 226.0 |
| MTHY04P | 4,511,080 | 4,487,410 (99.48%) | 415.7 |
| MTHY05P | 9,323,867 | 9,281,369 (99.54%) | 771.7 |
| MTHY06D | 6,834,346 | 6,659,238 (97.4%) | 284.6 |
| MTHY07P | 8,829,582 | 8,803,087 (99.7%) | 359.1 |
| MTHY08D | 7,066,657 | 6,968,076 (98.6%) | 506.7 |
| MTHY09P | 6,593,316 | 6,575,547 (99.7%) | 532.7 |
| MTHY10P | 7,176,855 | 7,147,576 (99.6%) | 473.6 |
| MTHY11D | 7,003,918 | 6,936,988 (99.0%) | 425.4 |
| MTHY12D | 6,363,318 | 6,302,508 (99.0%) | 327.7 |
| MTHY13P | 5,995,759 | 5,936,562 (99.01%) | 396.2 |
| MTHY14D | 6,693,272 | 6,669,570 (99.7%) | 483.5 |
| MTHY15P | 11,464,045 | 11,430,546 (99.71%) | 1272.9 |
| MTHY16D | 5,955,958 | 5,938,401 (99.7%) | 430.0 |
| MTHY17P | 17,119,682 | 17,044,262 (99.56%) | 993.6 |
| MTHY18D | 5,828,565 | 5,714,961 (98.1%) | 297.5 |
| MTHY19P | 8,536,959 | 8,505,742 (99.6%) | 574.8 |
| MTHY20D | 5,051,959 | 5,032,310 (99.6%) | 331.2 |
| MTHY21P | 10,922,522 | 10,819,754 (99.06%) | 554.3 |
| MTHY22D | 5,559,845 | 5,441,850 (97.9%) | 284.2 |
| MTHY23D | 5,812,076 | 5,771,019 (99.3%) | 231.5 |
| MTHY24D | 12,248,554 | 12,205,955 (99.7%) | 874.1 |
| MTHY25D | 8,227,847 | 8,177,699 (99.4%) | 578.7 |
| MTHY26P | 6,511,594 | 6,427,145 (98.7%) | 194.8 |
| MTHY27D | 7,687,089 | 7,600,913 (98.9%) | 331.9 |
| MTHY28P | 5,802,792 | 5,765,695 (99.4%) | 295.8 |
| MTHY29P | 7,559,034 | 7,460,946 (98.7%) | 200.5 |
| MTHY30P | 6,852,353 | 6,783,554 (99.0%) | 125.4 |
| MTHY31P | 4,704,611 | 4,667,408 (99.2%) | 146.8 |
| MTHY32P | 5,819,574 | 5,704,851 (98.0%) | 159.0 |
| MTHY33P | 5,090,963 | 5,070,051 (99.6%) | 273.8 |
| MTHY34P | 3,549,543 | 3,509,216 (98.9%) | 104.0 |
| MTHY35P | 3,966,836 | 3,774,090 (95.1%) | 120.7 |
| MTHY36P | 22,500,776 | 21,767,132 (96.7%) | 172.2 |
| MTHY37P | 18,584,333 | 18,577,774 (99.9%) | 857.8 |
| MTHY38P | 9,698,343 | 9,658,123 (99.6%) | 427.9 |
| MTHY39P | 16,075,669 | 15,717,320 (97.8%) | 274.0 |
| MTHY40D | 12,713,179 | 12,286,191 (96.6%) | 220.7 |
| MTHY41D | 23,440,449 | 23,282,804 (99.3%) | 463.6 |
| MTHY42P | 12,204,444 | 12,105,503 (99.2%) | 503.4 |
| MTHY43P | 13,568,319 | 13,386,065 (98.7%) | 240.7 |
| MTHY44P | 13,288,594 | 13,282,649 (99.9%) | 636.8 |
| MTHY45P | 14,362,238 | 14,143,106 (98.5%) | 425.9 |
| MTHY46P | 13,714,944 | 13,146,687 (95.9%) | 153.2 |
| MTHY47P | 13,620,199 | 13,589,937 (99.8%) | 1064.2 |
| MTHY48P | 8,987,607 | 8,878,946 (98.79%) | 272.2 |
| MTHY49P | 9,824,130 | 9,804,103 (99.8%) | 740.4 |
| MTHY50P | 14,147,721 | 14,001,842 (98.97%) | 181.6 |
| MTHY51P | 16,366,390 | 15,996,427 (97.74%) | 175.4 |
| MTHY52P | 11,160,337 | 11,126,546 (99.7%) | 518.2 |
| MTHY53P | 13,615,106 | 13,571,666 (99.68%) | 593.5 |
| MTHY54P | 13,583,087 | 13,532,225 (99.63%) | 1085.1 |
| MTHY55P | 12,449,381 | 12,403,489 (99.63%) | 549.3 |
| MTHY56P | 11,970,701 | 11,931,947 (99.68%) | 1222.1 |
| MTHY57P | 8,642,482 | 8,617,682 (99.71%) | 1093.1 |
| MTHY58P | 9,036,498 | 9,005,878 (99.66%) | 824.6 |
| MTHY59P | 8,560,447 | 8,535,971 (99.71%) | 860.3 |
| MTHY60P | 10,141,981 | 10,122,034 (99.8%) | 1188.7 |
| MTHY61P | 9,407,656 | 9,340,909 (99.29%) | 341.5 |

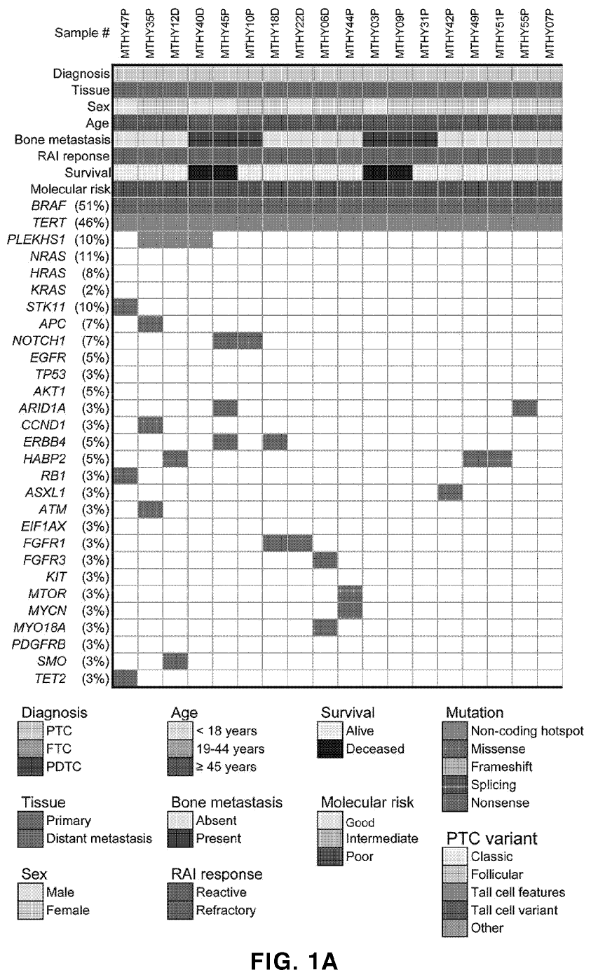

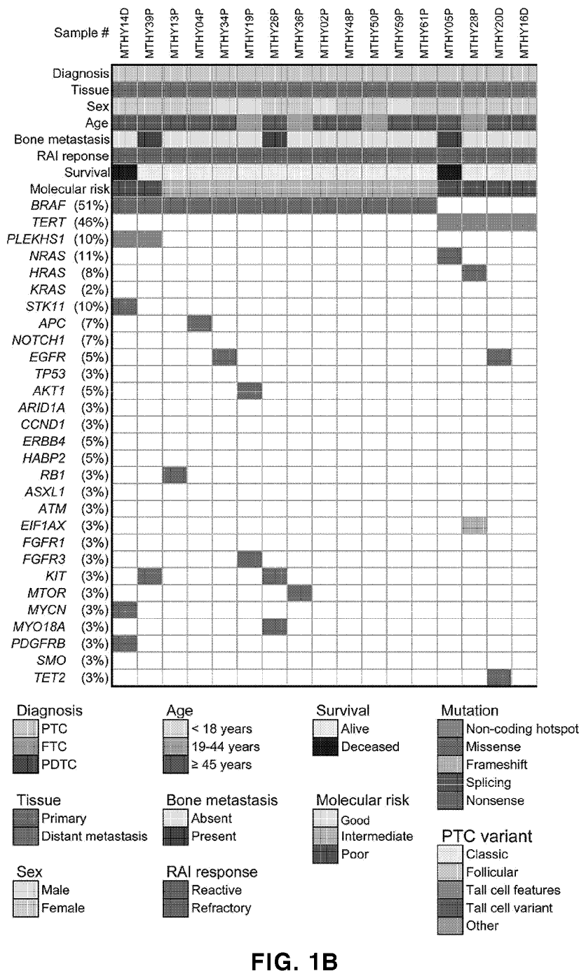

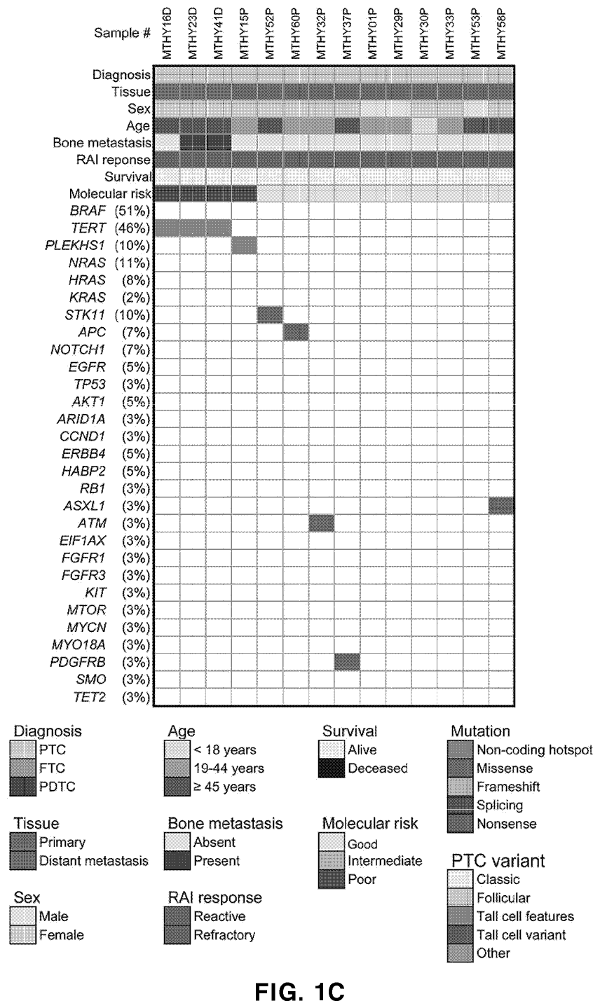

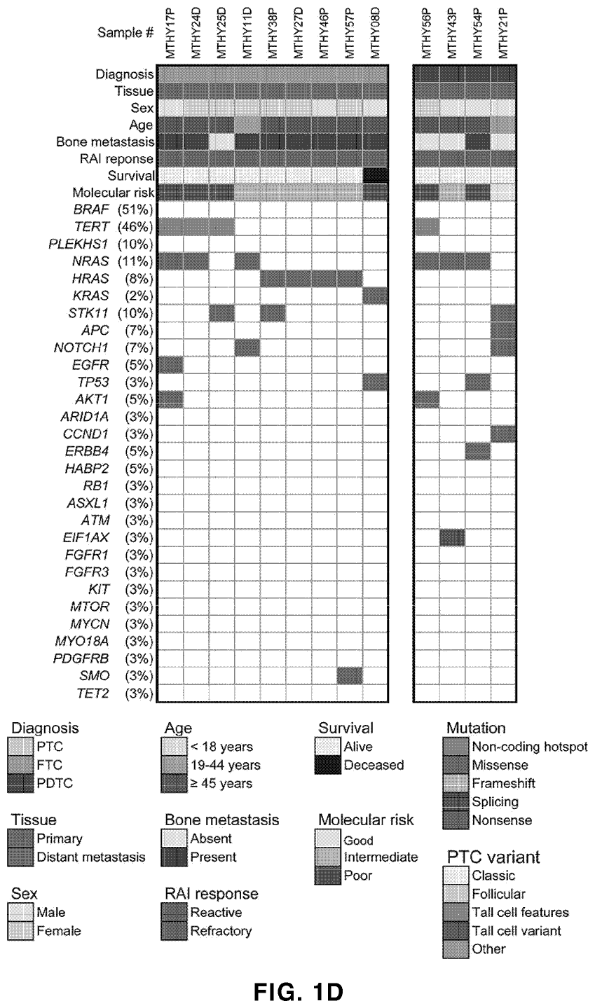

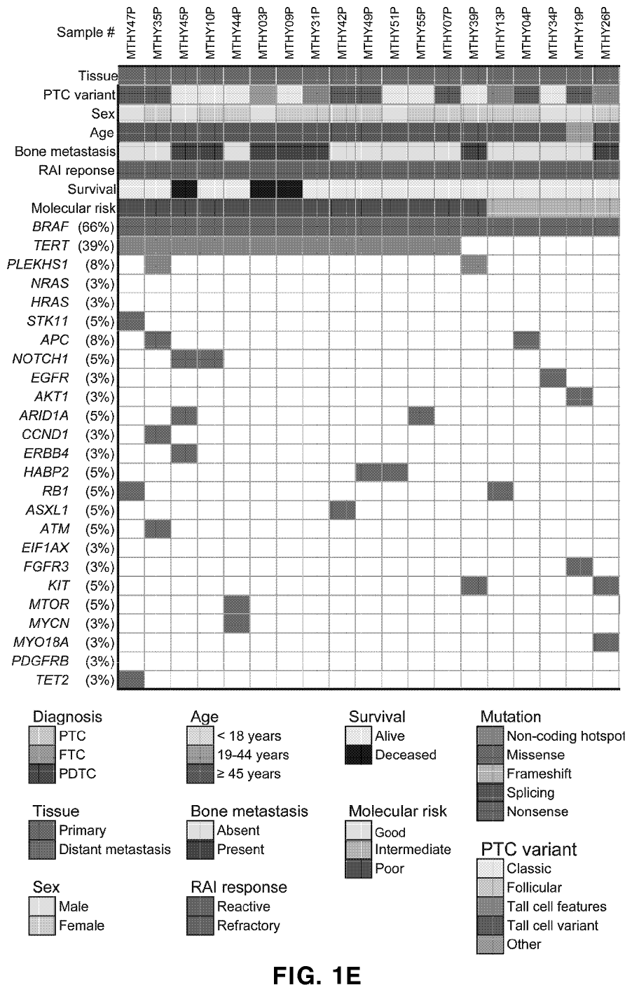

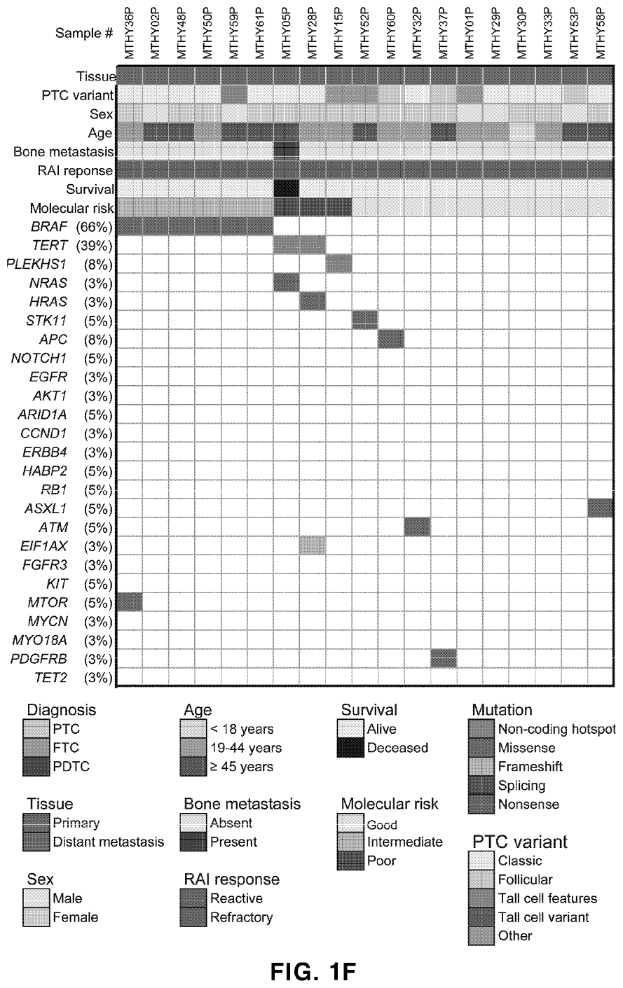

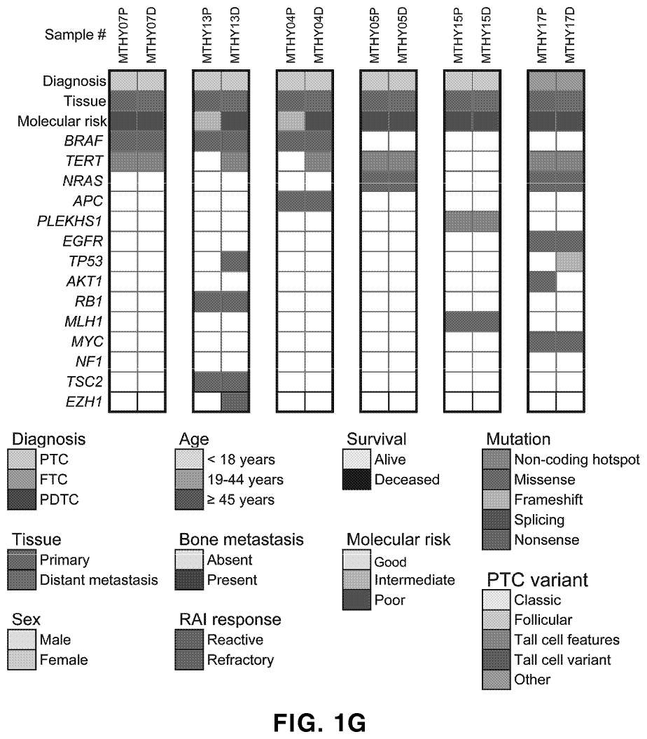

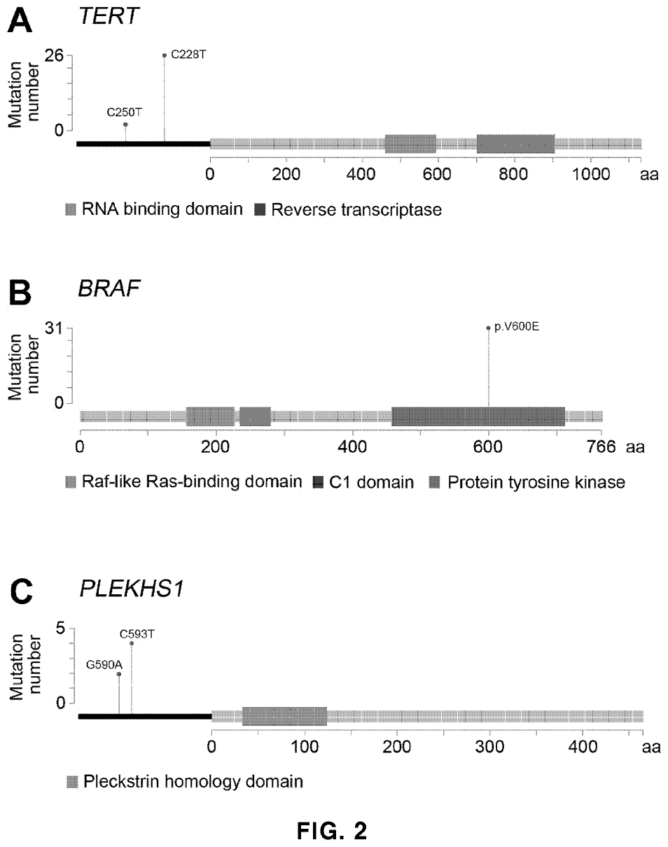

As illustrated in [FIG. 1A] to [FIG. 1G], a total of 203 somatic mutations (201 SNVs and 2 insertions-deletions) were confirmed, and most of the metastatic differentiated thyroid cancers (58/61, 95%) had non-silent mutations in at least one target gene. Of the mutated gene, mutations in the five genes BRAF (51%), the TERT promoter (46%), NRAS (13%), the PLEKHS1 promoter (10%) and STK11 (10%) were detected in 10% or more of metastatic differentiated thyroid cancer. The mutations in the TERT promoter were found in 24 (50%) of 48 papillary thyroid carcinomas and 3 (33%) of 9 follicular thyroid carcinomas. Further, as illustrated in [FIG. 2], among the mutations in the TERT promoter, the most common type is C228T (n=26, 43%), followed by C250T (n=2, 3%) (FIG. 2A).

Among the mutations in BRAF, p.V600E was the most predominant mutation in BRAF (FIG. 2 B) and was found only in papillary thyroid carcinomas (n=31, 65% of papillary thyroid carcinomas).

The mutation in the PLEKHS1 promoter was detected in 6 (13%, 6/48) patients with papillary thyroid carcinomas and was confirmed only in papillary thyroid carcinomas. All six mutations in the PLEKHS1 promoter were located at well-known hotspot sites (C593T, n=4 and G590A, n=2) (FIG. 2C). Of the 6 patients, 3 had coexisting mutations in BRAF and TERT promoters, 2 had mutations in BRAF, and 1 had no coexisting mutation.

All RAS mutations were detected in hotspot codon 61: one NRAS p.Q61R and one HRAS p.Q61R in two papillary thyroid carcinomas; three NRAS p.Q61Rs, four HRAS p.Q61Rs and one KRAS p.Q61R in eight follicular thyroid carcinomas; and two NRAS p.Q61Rs and one NRAS p.Q6K in three poorly differentiated thyroid carcinomas (FIGS. 1A to 1D).

Six STK11 mutations were found in three papillary thyroid carcinomas, two follicular thyroid carcinomas and one poorly differentiated thyroid carcinoma, and were all p.F354Ls (FIGS. 1A to 1D).

In addition to the 7 genes, mutations of TP53 (n=2; p.M237I, p.S240G), AKT1 (n=3; p.E17K), EIF1AX (n=2; p.A113fs, c.338-1G>A), PIK3CA (n=1; p.E545K and p.E726K double mutations) or TSHR (n=1; p.A623V) genes and the like were detected in metastatic differentiated thyroid cancer.

Mutation types could be analyzed in 38 primary papillary thyroid carcinomas, and the most common mutation was BRAF (n=25, 66%), followed by the TERT promoter (n=15, 39%) and the PLEKHS1 promoter (n=3, 8%) (FIGS. 1E and 1F).

When a mutual comparative analysis was performed on six cases of primary and metastatic differentiated thyroid cancers (five papillary thyroid carcinomas and one follicular thyroid carcinoma), 14 gene mutations were found (FIG. 1G). Among them, 10 coincided in both primary and metastatic differentiated thyroid cancers. However, in two cases (MTHY04 and MTHY13), mutations in TERT and TP53 were not found in primary tumors, but were found in metastatic tumor tissue. In the remaining one case (MTHY17P), the AKT1 mutation was found in primary tumor tissue, but was not found in metastatic tissue. The EZH1 mutation was found only in metastatic tumor tissue of one case (MTHY13).

### EXAMPLE 3

Verification of Validity as Distant Metastatic Marker for Mutations

Using a digital PCR device, the present inventors intended to verify the validity of targeted deep sequencing by verifying the non-coding hotspot mutation of the PLEKHS 1 gene detected by targeted deep sequencing. The primers and probes used for the verification are shown in the following [Table 4].

TABLE 4

| | Object to be | | SEQ ID |
|---|---|---|---|
| Target | applied | Primer (F, R) or probe (P) sequence | NO |
| PLEKHS1 G590A | Digital PCR | F: CCAAGGCTGGGATGATCTAGAAG | 1 |
| PLEKHS1 G590A | Digital PCR | R: AGCATATCTGCAAAATTTTCCATTTCCA | 2 |
| PLEKHS1 G590A | Digital PCR | P: CTTTTTTGCAATT[G/A]AACAAT | 3 |
| PLEKHS1 C593T | Digital PCR | F: GGCTGGGATGATCTAGAAGCTTTT | 4 |
| PLEKHS1 C593T | Digital PCR | R: AAGTGCCCATAACAGAAATACAGCATA | 5 |
| PLEKHS1 C593T | Digital PCR | P: ATTGAA[C/T]AATTGCAAAATTG | 6 |
| TERT | Sanger sequencing | F1: AGT GGA TTC GCG GGC ACA GA | 7 |
| TERT | Sanger sequencing | RI: CAG CGC TGC CTG AAA CTC | 8 |
| TERT | Sanger sequencing | F2: CAC CCG TCC TGC CCC TTC ACC TT | 9 |
| TERT | Sanger sequencing | R2: GGC TTC CCA CGT GCG CAG CAG GA | 10 |
| TERT | Sanger sequencing | F3: GTC CTG CCC CTT CAC CTT | 11 |
| TERT | Sanger sequencing | R3: CAG CGC TGC CTG AAA CTC | 12 |
| BRAF exon 15 | Sanger sequencing | F: TCA TAA TGC TTG CTC TGA TAG GA | 13 |
| BRAF exon 15 | Sanger sequencing | R: GGC CAA AAA TTT AAT CAG TGG A | 14 |
| NRAS codon 61 | Sanger sequencing | F: CCC CTT ACC CTC CAC ACC | 15 |
| NRAS codon 61 | Sanger sequencing | R: GAG GTT AAT ATC CGC AAA TGA CTT | 16 |
| NRAS codon 12, | Sanger sequencing | F: CTTGCTGGTGTGAAATGACTG | 17 |
| 13 | | | |
| NRAS codon 12, | Sanger sequencing | R: TCCGACAAGTGAGAGACAGG | 18 |
| 13 | | | |
| HRAS codon 61 | Sanger sequencing | F: GTC CTC CTG CAG GAT TCC TA | 19 |
| HRAS codon 61 | Sanger sequencing | R: CGG GGT TCA CCT GTA CT | 20 |
| HRAS codon 12, | Sanger sequencing | F: CTGAGGAGCGATGACGGAA | 21 |
| 13 | | | |
| HRAS codon 12, | Sanger sequencing | R: AGGCTCACCTCTATAGTGGG | 22 |
| 13 | | | |
| KRAS codon 61 | Sanger sequencing | F1: GGTGCACTGTAATAATCCAGAC | 23 |
| KRAS codon 61 | Sanger sequencing | R1: TGATTTAGTATTATTTATGGC | 24 |
| KRAS codon 61 | Sanger sequencing | F2: TGAAGTAAAAGGTGCACTGTAATA | 25 |
| KRAS codon 61 | Sanger sequencing | R2: TAAACCCACCTATAATGGTGAA | 26 |
| KRAS codon 12, | Sanger sequencing | F: GGT GAG TTT GTA TTA AAA GGT ACT | 27 |
| 13 | | GG | |
| KRAS codon 12, | Sanger sequencing | R: TCC TGC ACC AGT AAT ATG CA | 28 |
| 13 | | | |
| STK11 exonl | Sanger sequencing | F: CCGTTGGCACCCGTGACCTA | 29 |
| STK11 exonl | Sanger sequencing | R: ACCATCAGCACCGTGACTGG | 30 |
| STK11 exon2 | Sanger sequencing | F: GGGCGGATCACAAGGTCA | 31 |
| STK11 exon2 | Sanger sequencing | R: AGGAGACGGGAAGAGGAGC | 32 |
| STK11 exon3 | Sanger sequencing | F: TGTGCCCAGAGCAAGAGC | 33 |
| STK11 exon3 | Sanger sequencing | R: GCAGAAGAATGGCGTGAACC | 34 |
| STK11 exon4 & 5 | Sanger sequencing | F: AGGAGACGGGAAGAGGAGC | 35 |
| STK11 exon4 & 5 | Sanger sequencing | R: TGAACCACCATCTGCCGTAT | 36 |
| STK11 exon6 | Sanger sequencing | F: TGACTGACCACGCCTTTCTT | 37 |
| STK11 exon6 | Sanger sequencing | R: TGAGGGACCTGGCAAACC | 38 |
| STK11 exon7 | Sanger sequencing | F: CAGGGTCTGTCAGGGTTGTCC | 39 |
| STK11 exon7 | Sanger sequencing | R: CCGTCCGCTGCTCTGTCTT | 40 |
| STK11 exon8 | Sanger sequencing | F: ACTGCTTCTGGGCGTTTGC | 41 |
| STK11 exon8 | Sanger sequencing | R: AGGTGGGCTGGAGGCTTT | 42 |
| STK11 exon9 | Sanger sequencing | F: GGTTCTGTGCTGGCATTTCG | 43 |
| STK11 exon9 | Sanger sequencing | R: GGCTCTGACGCTGGTGGAT | 44 |
| STK11 exon 10a | Sanger sequencing | F: TGCCCAGGCTGACCTCTTC | 45 |
| STK11 exon 10a | Sanger sequencing | R: CGATGGCGTTTCTCGTGTTTT | 46 |
| STK11 exon 10b | Sanger sequencing | F: GGATTTGAGCTGTGGCTGTGAG | 47 |
| STK11 exon 10b | Sanger sequencing | R: AACACCGTGACTGCCGACCT | 48 |
| TP53 Exon 2, 3, 4 | Sanger sequencing | F: | 49 |
| | | TGTAAAACGACGGCCAGTGCCGAGCTGTCT | |
| | | CAGACAC | |
| TP53 Exon 2, 3, 4 | Sanger sequencing | R: | 50 |
| | | CAGGAAACAGCTATGACCGAAATGCAGGG | |
| | | GGATACG | |
| TP53 Exon 2, 3 | Sanger sequencing | F: | 51 |
| | | TGTAAAACGACGGCCAGTGGAGTGCTTGG | |
| | | GTTGTGGT | |
| TP53 Exon 2, 3 | Sanger sequencing | R: CAG GAA ACA GCT ATG ACC CGG CAA | 52 |
| | | GGG GGA CTG TA | |
| TP53 Exon 4 | Sanger sequencing | F: | 53 |
| | | TGTAAAACGACGGCCAGTGACTTCCTGAA | |
| | | AACAACG | |
| TP53 Exon 4 | Sanger sequencing | R: | 54 |
| | | CAGGAAACAGCTATGACCCACACATTAAG | |
| | | TGGGTAAAC | |
| TP53 Exon 5, 6 | Sanger sequencing | F: | 55 |
| | | TGTAAAACGACGGCCAGTTTTCTTTGCTGC | |
| | | CGTCTTC | |
| TP53 Exon 5, 6 | Sanger sequencing | R: | 56 |
| | | CAGGAAACAGCTATGACCTTGCACATCTCA | |
| | | TGGGGTTA | |
| TP53 Exon 7 | Sanger sequencing | F: | 57 |
| | | TGTAAAACGACGGCCAGTGACCATCCTGG | |
| | | CTAACGG | |
| TP53 Exon 7 | Sanger sequencing | R: | 58 |
| | | CAGGAAACAGCTATGACCCACAGGTTAAG | |
| | | AGGTCCCAAA | |
| TP53 Exon 8, 9 | Sanger sequencing | F: | 59 |
| | | TGTAAAACGACGGCCAGTTTTGGGACCTCT | |
| | | TAACCTGT | |
| TP53 Exon 8, 9 | Sanger sequencing | R: | 60 |
| | | CAGGAAACAGCTATGACCCAGGCAAAGTC | |
| | | ATAGAACCAT | |
| TP53 Exon 10 | Sanger sequencing | F: | 61 |
| | | TGTAAAACGACGGCCAGTCATGTTGCTTTT | |
| | | GTACCGTC | |
| TP53 Exon 10 | Sanger sequencing | R: | 62 |
| | | CAGGAAACAGCTATGACCGGCAAGAATGT | |
| | | GGTTATAGGA | |
| TP53 Exon 11 | Sanger sequencing | F: | 63 |
| | | TGTAAAACGACGGCCAGTAAGGGAAGATT | |
| | | ACGAGACT | |
| TP53 Exon 11 | Sanger sequencing | R: | 64 |
| | | CAGGAAACAGCTATGACCTAAGCTGGTAT | |
| | | GTCCTACTC | |
| TP53_cDNA_1 | Sanger sequencing | F: TCGGGCTGGGAGCGTGCTTT | 65 |
| TP53_cDNA_1 | Sanger sequencing | R: AGCTGCACAGGGCAGGTCTT | 66 |
| TP53_cDNA_2 | Sanger sequencing | F: GGACAGCCAAGTCTGTGACT | 67 |
| TP53_cDNA_2 | Sanger sequencing | R: GGAGAGGAGCTGGTGTTGTT | 68 |
| TP53_cDNA_3 | Sanger sequencing | F: CCATCCTCACCATCATCACA | 69 |
| TP53_cDNA_3 | Sanger sequencing | R: GCTGTCAGTGGGGAACAAGAA | 70 |

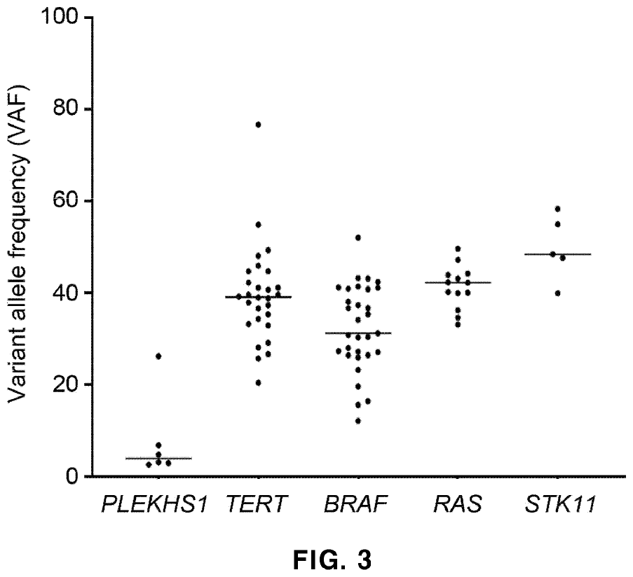

Digital PCR was performed using the TaqMan Genotyping assay QuantStudio 3D digital PCR system (Life Technologies), and 10 ng of genomic DNA was amplified with the TaqMan genotyping probe. For each assay, reference and mutant alleles were labeled with VIC and FAM dyes, respectively. After the alleles were labeled with the Nanofluidic chip, the raw data was analyzed using the Rare Mutation module of the QuantiStudio 3D AnalysisSuite Cloud Software. To confirm the mutation in the PLEKHS1 promoter confirmed by targeted deep sequencing, the present inventors performed digital PCR. As a result, as illustrated in [FIG. 3], most (5/6) of the mutations in the PLEKHS1 promoter occurred at a low (range: 2.6 to 6.8%) variant allele frequency (VAF). The average VAF (median 4.0%) of the mutation in the PLEKHS1 promoter was significantly lower than the other mutations detected in 10% or more of metastatic differentiated thyroid cancers. The results of digital PCR also showed similar VAFs for all mutations, similar to the results of targeted deep sequencing (Table 5).

TABLE 5

| | | VAF | |
|---|---|---|---|
| | | Targeted deep | Digital |
| Sample ID | Mutation | sequencing | PCR |
| MTHY12 | C593T | 3.1% | 3.7% |
| MTHY14 | C593T | 26.2% | 21.9% |
| MTHY15 | C593T | 2.6% | 3.9% |
| MTHY35 | G590A | 4.8% | 5.9% |
| MTHY39 | C593T | 6.8% | 4.1% |
| MTHY40 | G590A | 2.9% | 2.0% |

Next, the present inventors performed digital PCR on independent patients (n=75) with papillary thyroid carcinoma and mutations in the PLEKHS1 promoter without any distant metastasis during the initial surgery of primary tumors in order to confirm whether the mutation in the PLEKHS1 promoter was a metastasis-specific event. The mutation in the PLEKHS1 promoter was found in only one case (1/75, 1.3%), and the tumor thereof showed aggressive histology (hobnail variant), thyroid dilatation and central lymph node metastasis. As a result of examining a 4-year history of this mutation-positive case after the initial surgery, various metastatic nodules appeared in both lungs 3 years after thyroidectomy on chest computed tomography, supporting the hypothesis that the mutation in the PLEKHS1 promoter is a metastasis-associated event. Except for this case, no distant metastasis was observed in the other 74 cases.

In the current study, most (5/6) of the mutations in the PLEKHS1 promoter show low VAF (range: 2.6 to 6.8%), suggesting that the gain of the mutations in the PLEKHS1 promoter may be a later event during tumor progression.

### EXAMPLE 4

Copy Number Variation in Metastatic Thyroid Cancer Gene

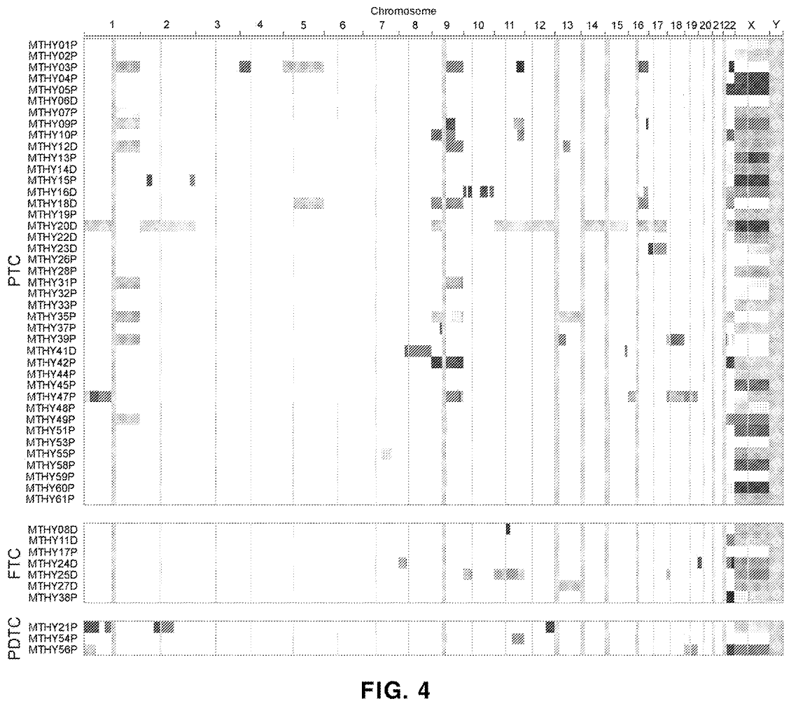

The copy number variation (CNV) and loss of heterozygosity (LOH) profiles of 51 metastatic thyroid cancers were analyzed using targeted deep sequencing analysis data (FIG. 4).

Copy number variation profiling was calculated by analyzing the data of targeted deep sequencing. The copy number variation in each sample was analyzed using the multiscale reference module and SNP Rank Segmentation statistical algorithm present in the NEXUS software v10 (Biodiscovery, El Segundo, Calif.). The segment was classified as gain when the log 2 ratio was greater than 0.18 and loss when it was less than −0.018. All confirmed copy number variations were manually confirmed directly.

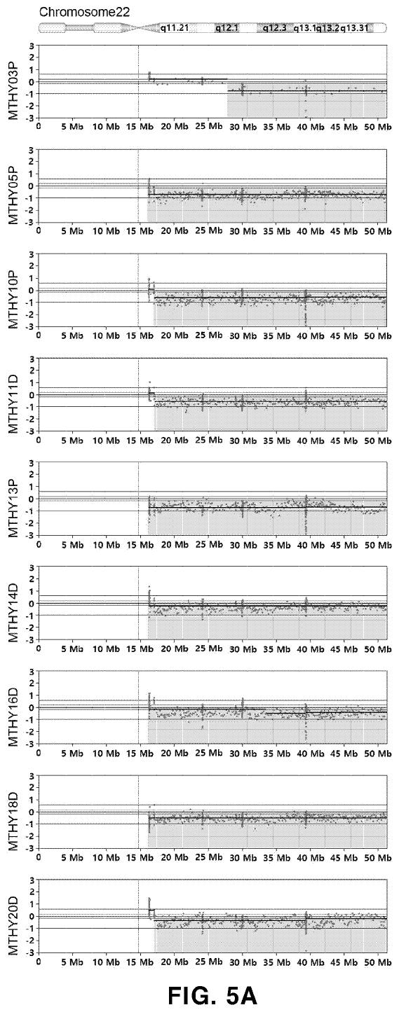

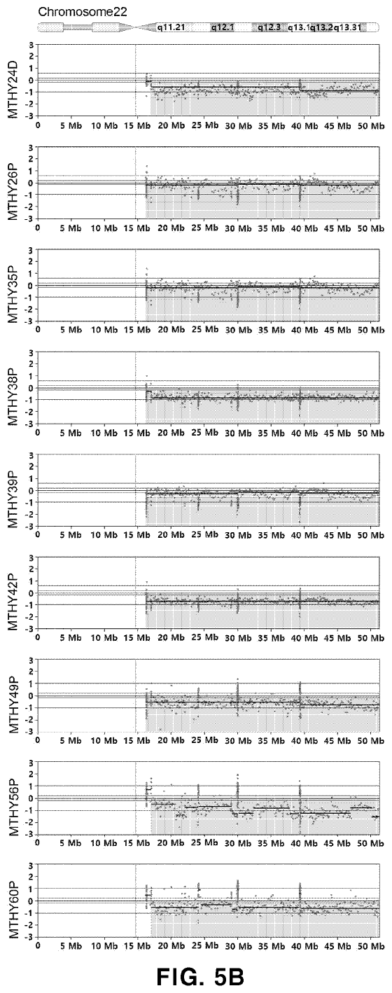

10 cases were excluded due to high background noise levels. Of the 106 confirmed copy number variations (Table 6), 22 copy number variations repeatedly appeared (>2 cases) (Table 7). The highest recurrent (18/51, 35%) copy number variation was the loss of the 35 Mb sized 22q11.1-q13.33 regions including NF2, EP300 and CHEK2 genes (FIGS. 5A and 5B). 14 (34%) of 41 papillary thyroid carcinomas, 3 (43%) of 7 follicular thyroid carcinomas and 1 (33%) of 3 undifferentiated thyroid carcinomas had this loss. Repeated copy number loss of 9p24.3-p11.2, 9q12-q34.3, and 16q11.2-q24.3 and gain of 1q12-q44 were detected only in papillary thyroid carcinomas, and the frequencies thereof were 15% (n=6), 22% (n=9) and 12% (n=5), respectively, in 41 papillary thyroid carcinomas. The following [Table 6] shows the copy number variation results analyzed in 40 cases of differentiated thyroid cancer accompanied by distant metastasis. The following [Table 7] shows repeated regions of the copy number variation.

TABLE 6

| | | | Chromosome | |
|---|---|---|---|---|
| Sample ID | Position | Event | position | Oncogene examination |
| MTHY03P | chr1:143,667,911- | Gain | q21.1-q44 | PDE4DIP, BCL9, ARNT, TPM3, MUC1, PRCC, |
| | 249,250,621 | | | NTRK1, SDHC, FCGR2B, PBX1, ABL2, TPR, |
| | | | | MDM4, ELK4, SLC45A3, H3F3A, FH |
| MTHY03P | chr5:0- | Gain | p15.33-p12 | IL7R, LIFR |
| | 45,048,530 | | | |
| MTHY03P | chr5:49,448,731- | Gain | q11.1-q35.3 | IL6ST, PIK3R1, APC, PDGFRB, CD74, ITK, |
| | 180,915,260 | | | EBF1, RANBP17, TLX3, NPM1, NSD1 |
| MTHY03P | chr4:0- | Loss | p16.3-q11 | FGFR3, WHSC1, SLC34A2, PHOX2B |
| | 50,400,000 | | | |
| MTHY03P | chr9:66,971,216- | Loss | q13-q34.3 | GNAQ, SYK, OMD, FANCC, XPA, NR4A3, |
| | 141,213,431 | | | TAL2, SET, FNBP1, ABL1, NUP214, TSC1, |
| | | | | RALGDS, BRD3, NOTCH1 |
| MTHY03P | chr11:100,048,708- | Loss | q22.1-q25 | BIRC3, ATM, DDX10, POU2AF1, SDHD, |
| | 135,006,516 | | | PAFAH1B2, PCSK7, MLL, DDX6, CBL, |
| | | | | ARHGEF12, FLI1 |
| MTHY03P | chr16:46,391,627- | Loss | q11.2-q24.3 | CYLD, HERPUD1, CDH11, CBFB, CDH1, MAF, |
| | 90,354,753 | | | CBFA2T3, FANCA |
| MTHY03P | chr22:27,894,333- | Loss | q12.1-q13.33 | MN1, CHEK2, EWSR1, NF2, MYH9, PDGFB, |
| | 51,304,566 | | | MKL1, EP300 |
| MTHY05P | chr22:17,058,341- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY07P | chr1:142,563,911- | Gain | q12-q44 | PDE4DIP, BCL9, ARNT, TPM3, MUC1, PRCC, |
| | 249,250,621 | | | NTRK1, SDHC, FCGR2B, PBX1, ABL2, TPR, |
| | | | | MDM4, ELK4, SLC45A3, H3F3A, FH |
| MTHY08D | chr11:54,956,509- | Loss | q11-q13.3 | MEN1 |
| | 68,722,608 | | | |
| MTHY08D | chr11:68,722,608- | Gain | q13.3-q13.4 | CCND1, NUMA1 |
| | 72,185,108 | | | |
| MTHY09P | chr1:142,563,911- | Gain | q12-q44 | PDE4DIP, BCL9, ARNT, TPM3, MUC1, PRCC, |
| | 249,250,621 | | | NTRK1, SDHC, FCGR2B, PBX1, ABL2, TPR, |
| | | | | MDM4, ELK4, SLC45A3, H3F3A, FH |
| MTHY09P | chr9:65,605,116- | Loss | q12-q31.1 | GNAQ, SYK, OMD, FANCC, XPA, NR4A3 |
| | 104,748,015 | | | |
| MTHY09P | chr11:89,781,008- | Loss | q14.3-q25 | MAML2, BIRC3, ATM, DDX10, POU2AF1, |
| | 135,006,516 | | | SDHD, PAFAH1B2, PCSK7, MLL, DDX6, CBL, |
| | | | | ARHGEF12, FLI1 |
| MTHY09P | chr16:81,090,226- | Loss | q23.2-q24.3 | CBFA2T3, FANCA |
| | 90,354,753 | | | |
| MTHY10P | chr9:0- | Loss | p24.3-p11.2 | JAK2, CD274, NFIB, MLLT3, FANCG, PAX5 |
| | 47,174,615 | | | |
| MTHY10P | chr9:65,605,116- | Loss | q12-q31.1 | GNAQ, SYK, OMD, FANCC, XPA, NR4A3 |
| | 105,985,615 | | | |
| MTHY10P | chr11:106,203,508- | Loss | q22.3-q25 | ATM, DDX10, POU2AF1, SDHD, PAFAH1B2, |
| | 135,006,516 | | | PCSK7, MLL, DDX6, CBL, ARHGEF12, FLI1 |
| MTHY10P | chr22:17,055,433- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY11D | chr22:17,054,883- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY03P | chr1:143,667,911- | Gain | q21.1-q44 | PDE4DIP, BCL9, ARNT, TPM3, MUC1, PRCC, |
| | 249,250,621 | | | NTRK1, SDHC, FCGR2B, PBX1, ABL2, TPR, |
| | | | | MDM4, ELK4, SLC45A3, H3F3A, FH |
| MTHY12D | chr9:65,605,116- | Loss | q12-q34.3 | GNAQ, SYK, OMD, FANCC, XPA, NR4A3, |
| | 141,213,431 | | | TAL2, SET, FNBP1, ABL1, NUP214, TSC1, |
| | | | | RALGDS, BRD3, NOTCH1 |
| MTHY12D | chr13:38,643,839- | Loss | q13.3-q21.33 | LHFP, LCP1, RB1 |
| | 70,914,139 | | | |
| MTHY13P | chr22:16,205,534- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY14D | chr16:2,494,626- | Loss | p13.3-p11.1 | CREBBP, CIITA, SOCS1, TNFRSF17, ERCC4, |
| | 35,239,726 | | | MYH11, PALB2, IL21R, FUS |
| MTHY14D | chr22:16,205,534- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY15P | chr2:29,993,569- | Loss | p23.2-p16.2 | ALK, EML4, MSH2, MSH6, FBXO11 |
| | 53,684,636 | | | |
| MTHY15P | chr2:222,034,436- | Loss | q36.1-q37.3 | PAX3, ACSL3 |
| | 243,199,373 | | | |
| MTHY16D | chr10:0- | Loss | p15.3-p13 | GATA3 |
| | 13,570,723 | | | |
| MTHY16D | chr10:21,815,323- | Loss | p12.31-p11.1 | MLLT10, KIF5B |
| | 39,140,823 | | | |
| MTHY16D | chr10:73,957,123- | Loss | q22.1-q25.1 | BMPR1A, FAM22A, PTEN, TLX1, NFKB2, |
| | 108,768,623 | | | SUFU |
| MTHY16D | chr10:118,277,323- | Loss | q25.3-q26.3 | FGFR2, DUX4 |
| | 135,534,747 | | | |
| MTHY16D | chr16:69,705,326- | Loss | q22.1-q24.3 | MAF, CBFA2T3, FANCA |
| | 90,354,753 | | | |
| MTHY16D | chr22:16,205,534- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY18D | chr5:49,437,731- | Gain | q11.1-q35.3 | IL6ST, PIK3R1, APC, PDGFRB, CD74, ITK, |
| | 180,915,260 | | | EBF1, RANBP17, TLX3, NPM1, NSD1 |
| MTHY18D | chr9:0- | Loss | p24.3-p11.2 | JAK2, CD274, NFIB, MLLT3, FANCG, PAX5 |
| | 47,174,615 | | | |
| MTHY18D | chr9:65,605,116- | Loss | q12-q34.3 | GNAQ, SYK, OMD, FANCC, XPA, NR4A3, |
| | 141,213,431 | | | TAL2, SET, FNBP1, ABL1, NUP214, TSC1, |
| | | | | RALGDS, BRD3, NOTCH1 |
| MTHY18D | chr16:46,391,327- | Loss | q11.2-q24.3 | CYLD, HERPUD1, CDH11, CBFB, CDH1, MAF, |
| | 90,354,753 | | | CBFA2T3, FANCA |
| MTHY18D | chr22:16,205,534- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY20D | chr12:0- | Gain | P13.33-p11.1 | KDM5A, CCND2, ZNF384, ETV6, KRAS |
| | 34,763,247 | | | |
| MTHY20D | chr12:37,886,848- | Gain | q11-q24.33 | ARID2, MLL2, ATF1, HOXC13, HOXC11, |
| | 133,851,895 | | | NACA, DDIT3, CDK4, LRIG3, WIF1, HMGA2, |
| | | | | MDM2, BTG1, ALDH2, PTPN11, BCL7A |
| MTHY20D | chr14:19,064,221- | Gain | q11.1-q32.33 | CCNB1IP1, TRA@, NKX2-1, NIN, KTN1, |
| | 107,349,540 | | | GPHN, TSHR, TRIP11, GOLGA5, DICER1, |
| | | | | TCL6, TCL1A, BCL11B, AKT1, IGH@ |
| MTHY20D | chr17:0- | Gain | p13.3-p11.1 | YWHAE, USP6, TP53, PER1, GAS7, MAP2K4 |
| | 22,257,405 | | | |
| MTHY20D | chr17:25,281,306- | Gain | q11.1-q25.3 | NF1, SUZ12, TAF15, MLLT6, LASP1, CDK12, |
| | 81,195,210 | | | ERBB2, RARA, BRCA1, ETV4, COL1A1, HLF, |
| | | | | MSI2, CLTC, BRIP1, CD79B, DDX5, |
| | | | | PRKAR1A, SRSF2, CANT1, ASPSCR1 |
| MTHY20D | chr1:0- | Loss | p36.33-p11.2 | TNFRSF14, PRDM16, RPL22, CAMTA1, SDHB, |
| | 121,483,410 | | | PAX7, MDS2, ARID1A, LCK, SFPQ, THRAP3, |
| | | | | MYCL1, MPL, MUTYH, TAL1, CDKN2C, |
| | | | | EPS15, JUN, JAK1, FUBP1, BCL10, RBM15, |
| | | | | TRIM33, NRAS, FAM46C, NOTCH2 |
| MTHY20D | chr2:0- | Loss | p25.3-p11.1 | MYCN, C2orf44, NCOA1, DNMT3A, ALK, |
| | 90,536,136 | | | EML4, MSH2, MSH6, FBXO11, BCL11A, REL, |
| | | | | XPO1, IGL@ |
| MTHY20D | chr2:95,533,037- | Loss | q11.1-q37.3 | TTL, PAX8, ERCC3, CHN1, HOXD13, |
| | 243,199,373 | | | HOXD11, NFE2L2, PMS1, SF3B1, CREB1, |
| | | | | IDH1, ATIC, FEV, PAX3, ACSL3 |
| MTHY20D | chr9:0- | Loss | p24.3-p11.2 | JAK2, CD274, NFIB, MLLT3, FANCG, PAX5 |
| | 47,174,615 | | | |
| MTHY20D | chr11:0- | Loss | p15.5-p11.12 | HRAS, CARS, NUP98, LMO1, FANCF, WT1, |
| | 51,589,008 | | | LMO2, EXT2, CREB3L1, DDB2 |
| MTHY20D | chr11:54,956,509- | Loss | q11-q25 | MEN1, CCND1, NUMA1, PICALM, MAML2, |
| | 135,006,516 | | | BIRC3, ATM, DDX10, POU2AF1, SDHD, |
| | | | | PAFAH1B2, PCSK7, MLL, DDX6, CBL, |
| | | | | ARHGEF12, FLI1 |
| MTHY20D | chr15:22,525,346- | Loss | q11.2-q26.3 | C15orf55, BUB1B, FLJ27352, TCF12, PML, |
| | 102,531,392 | | | NTRK3, IDH2, CRTC3, BLM |
| MTHY20D | chr16:46,391,327- | Loss | q11.2-q24.3 | CYLD, HERPUD1, CDH11, CBFB, CDH1, MAF, |
| | 90,354,753 | | | CBFA2T3, FANCA |
| MTHY20D | chr22:17,054,883- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY21P | chr1:0- | Loss | p36.33-p31.3 | TNFRSF14, PRDM16, RPL22, CAMTA1, SDHB, |
| | 67,810,810 | | | PAX7, MDS2, ARID1A, LCK, SFPQ, THRAP3, |
| | | | | MYCL1, MPL, MUTYH, TAL1, CDKN2C, |
| | | | | EPS15, JUN, JAK1 |
| MTHY21P | chr1:92,363,710- | Loss | p22.1-p11.2 | RBM15, TRIM33, NRAS, FAM46C, NOTCH2 |
| | 121,483,410 | | | |
| MTHY21P | chr2:61,797,036- | Loss | p15-p11.1 | IGL@ |
| | 90,536,136 | | | |
| MTHY21P | chr2:95,533,037- | Loss | q11.1-q23.1 | TTL, PAX8, ERCC3 |
| | 149,082,436 | | | |
| MTHY21P | chr12:94,657,647- | Loss | q22-q24.33 | ALDH2, PTPN11, BCL7A |
| | 133,851,895 | | | |
| MTHY23D | chr17:25,281,306- | Gain | q11.1-q25.3 | NF1, SUZ12, TAF15, MLLT6, LASP1, CDK12, |
| | 81,195,210 | | | ERBB2, RARA, BRCA1, ETV4, COL1A1, HLF, |
| | | | | MSI2, CLTC, BRIP1, CD79B, DDX5, |
| | | | | PRKAR1A, SRSF2, CANT1, ASPSCR1 |
| MTHY23D | chr17:0- | Loss | p13.3-p11.1 | YWHAE, USP6, TP53, PER1, GAS7, MAP2K4 |
| | 22,257,405 | | | |
| MTHY24D | chr8:0- | Gain | p23.3-p11.22 | PCM1, WRN, WHSC1L1, FGFR1 |
| | 38,365,411 | | | |
| MTHY24D | chr20:0- | Loss | p13-p11.23 | |
| | 17,930,910 | | | |
| MTHY24D | chr22:17,055,433- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY25D | chr10:0- | Loss | p15.3-p11.1 | GATA3, MLLT10, KIF5B |
| | 39,140,823 | | | |
| MTHY25D | chr11:0- | Loss | p15.5-p11.12 | HRAS, CARS, NUP98, LMO1, FANCF, WT1, |
| | 51,589,008 | | | LMO2, EXT2, CREB3L1, DDB2 |
| MTHY25D | chr11:54,956,509- | Loss | q11-q25 | MEN1, CCND1, NUMA1, PICALM, MAML2, |
| | 135,006,516 | | | BIRC3, ATM, DDX10, POU2AF1, SDHD, |
| | | | | PAFAH1B2, PCSK7, MLL, DDX6, CBL, |
| | | | | ARHGEF12, FLI1 |
| MTHY25D | chr18:0- | Loss | p11.32- | |
| | 15,351,224 | | p11.21 | |
| MTHY26P | chr22:16,205,534- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY27D | chr13:19,168,940- | Loss | q11-q34 | CDX2, FLT3, BRCA2, LHFP, LCP1, RB1, |
| | 115,169,878 | | | ERCC5 |
| MTHY31P | chr1:142,563,911- | Gain | q12-q44 | PDE4DIP, BCL9, ARNT, TPM3, MUC1, PRCC, |
| | 249,250,621 | | | NTRK1, SDHC, FCGR2B, PBX1, ABL2, TPR, |
| | | | | MDM4, ELK4, SLC45A3, H3F3A, FH |
| MTHY31P | chr9:65,605,116- | Loss | q12-q34.3 | GNAQ, SYK, OMD, FANCC, XPA, NR4A3, |
| | 141,213,431 | | | TAL2, SET, FNBP1, ABL1, NUP214, TSC1, |
| | | | | RALGDS, BRD3, NOTCH1 |
| MTHY35P | chr1:142,563,911 | Gain | q12-q44 | PDE4DIP, BCL9, ARNT, TPM3, MUC1, PRCC, |
| | -249,250,621 | | | NTRK1, SDHC, FCGR2B, PBX1, ABL2, TPR, |
| | | | | MDM4, ELK4, SLC45A3, H3F3A, FH |
| MTHY35P | chr9:0- | Loss | p24.3-p11.2 | JAK2, CD274, NFIB, MLLT3, FANCG, PAX5 |
| | 47,174,615 | | | |
| MTHY35P | chr9:65,605,116- | Loss | q12-q34.3 | GNAQ, SYK, OMD, FANCC, XPA, NR4A3, |
| | 141,213,431 | | | TAL2, SET, FNBP1, ABL1, NUP214, TSC1, |
| | | | | RALGDS, BRD3, NOTCH1 |
| MTHY35P | chr13:19,168,940- | Loss | q11-q34 | CDX2, FLT3, BRCA2, LHFP, LCP1, RB1, |
| | 115,169,878 | | | ERCC5 |
| MTHY35P | chr22:16,205,534- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY37P | chr9:38,783,815- | Loss | p13.1-p11.2 | |
| | 47,174,615 | | | |
| MTHY38P | chr22:16,205,534- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY39P | chr1:142,563,911- | Gain | q12-q44 | PDE4DIP, BCL9, ARNT, TPM3, MUC1, PRCC, |
| | 249,250,621 | | | NTRK1, SDHC, FCGR2B, PBX1, ABL2, TPR, |
| | | | | MDM4, ELK4, SLC45A3, H3F3A, FH |
| MTHY39P | chr13:19,168,940- | Loss | q11-q14.2 | CDX2, FLT3, BRCA2, LHFP, LCP1, RB1 |
| | 49,589,739 | | | |
| MTHY39P | chr18:0- | Loss | p11.32- | |
| | 15,351,224 | | p11.21 | |
| MTHY39P | chr18:18,516,325- | Loss | q11.1-q23 | ZNF521, SS18, MALT1, BCL2 |
| | 78,077,248 | | | |
| MTHY39P | chr22:16,205,534- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY41D | chr8:29,947,911- | Gain | p12-p11.1 | WRN, WHSC1L1, FGFR1, HOOK3 |
| | 43,831,311 | | | |
| MTHY41D | chr8:46,845,512- | Gain | q11.1-q24.3 | TCEA1, PLAG1, CHCHD7, NCOA2, HEY1, |
| | 146,364,022 | | | COX6C, EXT1, MYC, NDRG1, RECQL4 |
| MTHY41D | chr15:89,006,346- | Gain | q25.3-q26.3 | IDH2, CRTC3, BLM |
| | 99,436,239 | | | |
| MTHY42P | chr9:0- | Loss | p24.3-p11.2 | JAK2, CD274, NFIB, MLLT3, FANCG, PAX5 |
| | 47,174,615 | | | |
| MTHY42P | chr9:65,605,116- | Loss | q12-q34.3 | GNAQ, SYK, OMD, FANCC, XPA, NR4A3, |
| | 141,213,431 | | | TAL2, SET, FNBP1, ABL1, NUP214, TSC1, |
| | | | | RALGDS, BRD3, NOTCH1 |
| MTHY42P | chr22:16,205,534- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY47P | chr1:0- | Loss | p36.33-p11.2 | TNFRSF14, PRDM16, RPL22, CAMTA1, SDHB, |
| | 121,483,410 | | | PAX7, MDS2, ARID1A, LCK, SFPQ, THRAP3, |
| | | | | MYCL1, MPL, MUTYH, TAL1, CDKN2C, |
| | | | | EPS15, JUN, JAK1, FUBP1, BCL10, RBM15, |
| | | | | TRIM33, NRAS, FAM46C, NOTCH2 |
| MTHY47P | chr9:65,605,116- | Loss | q12-q34.3 | GNAQ, SYK, OMD, FANCC, XPA, NR4A3, |
| | 141,213,431 | | | TAL2, SET, FNBP1, ABL1, NUP214, TSC1, |
| | | | | RALGDS, BRD3, NOTCH1 |
| MTHY47P | chr16:2,152,532- | Loss | p13.3-p11.1 | CREBBP, CIITA, SOCS1, TNFRSF17, ERCC4, |
| | 35,239,726 | | | MYH11, PALB2, IL21R, FUS |
| MTHY47P | chr18:0- | Loss | p11.32- | |
| | 15,351,224 | | p11.21 | |
| MTHY47P | chr18:18,516,325- | Loss | q11.1-q23 | ZNF521, SS18, MALT1, BCL2 |
| | 78,077,248 | | | |
| MTHY47P | chr19:0- | Loss | p13.3-p11 | FSTL3, STK11, TCF3, GNA11, SH3GL1, |
| | 24,617,641 | | | MLLT1, DNM2, SMARCA4, LYL1, BRD4, |
| | | | | TPM4, JAK3, ELL |
| MTHY47P | chr19:27,736,742- | Loss | q11-q13.43 | CCNE1, CEBPA, AKT2, CD79A, CIC, BCL3, |
| | 59,128,983 | | | CBLC, ERCC2, KLK2, PPP2R1A, ZNF331, TFPT |
| MTHY49P | chr1:143,575,460- | Gain | q21.1-q44 | PDE4DIP, BCL9, ARNT, TPM3, MUC1, PRCC, |
| | 249,250,621 | | | NTRK1, SDHC, FCGR2B, PBX1, ABL2, TPR, |
| | | | | MDM4, ELK4, SLC45A3, H3F3A, FH |
| MTHY49P | chr22:16,205,534- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY54P | chr11:82,040,300- | Loss | q14.1-q25 | PICALM, MAML2, BIRC3, ATM, DDX10, |
| | 135,006,516 | | | POU2AF1, SDHD, PAFAH1B2, PCSK7, MLL, |
| | | | | DDX6, CBL, ARHGEF12, FLI1 |
| MTHY55P | chr7:86,794,747- | Loss | q21.12-q32.1 | AKAP9, CDK6, MET |
| | 128,711,135 | | | |
| MTHY56P | chr1:1- | Loss | p36.33-p32.3 | TNFRSF14, PRDM16, RPL22, CAMTA1, SDHB, |
| | 55,951,352 | | | PAX7, MDS2, ARID1A, LCK, SFPQ, THRAP3, |
| | | | | MYCL1, MPL, MUTYH, TAL1, CDKN2C, |
| | | | | EPS15 |
| MTHY56P | chr19:1- | Loss | p13.3-p11 | FSTL3, STK11, TCF3, GNA11, SH3GL1, |
| | 24,617,641 | | | MLLT1, DNM2, SMARCA4, LYL1, BRD4, |
| | | | | TPM4, JAK3, ELL |
| MTHY56P | chr19:32,782,941- | Loss | q13.11- | CEBPA, AKT2, CD79A, CIC, BCL3, CBLC, |
| | 59,128,983 | | q13.43 | ERCC2, KLK2, PPP2R1A, ZNF331, TFPT |
| MTHY56P | chr22:17,055,533- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |
| MTHY60P | chr22:17,057,347- | Loss | q11.1-q13.33 | CLTCL1, BCR, SMARCB1, MN1, CHEK2, |
| | 51,304,566 | | | EWSR1, NF2, MYH9, PDGFB, MKL1, EP300 |

TABLE 7

| | Chromosome | | | PTC | FTC | PDTC | |
|---|---|---|---|---|---|---|---|
| Chromosome region | position | Event | Frequency | (n = 41) | (n = 7) | (n = 3) | Type |
| chr1:0-55,951,352 | p36.33-p32.3 | Loss | 4 | 2 | 0 | 2 | Mixed |
| chr1:55,951,353-67,810,810 | p32.3-p31.3 | Loss | 3 | 2 | 0 | 1 | Mixed |
| chr1:92,363,710-121,483,410 | p22.1-p11.2 | Loss | 3 | 2 | 0 | 1 | Mixed |
| chr1:142,563,911-249,250,621 | q12-q44 | Gain | 8 | 8 | 0 | 0 | PTC- |
| | | | | | | | specific |
| chr9:0-38,783,814 | p24.3-p13.1 | Loss | 5 | 5 | 0 | 0 | PTC- |
| | | | | | | | specific |
| chr9:38,783,815-47,174,615 | p13.1-p11.2 | Loss | 6 | 6 | 0 | 0 | PTC- |
| | | | | | | | specific |
| chr9:65,605,116-105,985,615 | q12-q31.1 | Loss | 9 | 9 | 0 | 0 | PTC- |
| | | | | | | | specific |
| chr9:105,985,615-141,213,431 | q31.1-q34.3 | Loss | 7 | 7 | 0 | 0 | PTC- |
| | | | | | | | specific |
| chr11:54,956,509-68,722,608 | q11-q13.3 | Loss | 3 | 1 | 2 | 0 | Mixed |
| chr11:82,040,300-89,781,007 | q14.1-q14.3 | Loss | 3 | 1 | 1 | 1 | Mixed |
| chr11:89,781,008-100,048,707 | q14.3-q22.1 | Loss | 4 | 2 | 1 | 1 | Mixed |
| chr11:100,048,708- | q22.1-q22.3 | Loss | 5 | 3 | 1 | 1 | Mixed |
| 106,203,507 | | | | | | | |
| chr11:106,203,508- | q22.3-q25 | Loss | 6 | 4 | 1 | 1 | Mixed |
| 135,006,516 | | | | | | | |
| chr13:19,168,940-38,643,838 | q11-q13.3 | Loss | 3 | 2 | 1 | 0 | Mixed |
| chr13:38,643,839-49,589,739 | q13.3-q14.2 | Loss | 4 | 3 | 1 | 0 | Mixed |
| chr13:49,589,740-70,914,139 | q14.2-q21.33 | Loss | 3 | 2 | 1 | 0 | Mixed |
| chr16:46,391,327-69,705,325 | q11.2-q22.1 | Loss | 3 | 3 | 0 | 0 | PTC- |
| | | | | | | | specific |
| chr16:69,705,326-81,090,225 | q22.1-q23.2 | Loss | 4 | 4 | 0 | 0 | PTC- |
| | | | | | | | specific |
| chr16:81,090,226-90,354,753 | q23.2-q24.3 | Loss | 5 | 5 | 0 | 0 | PTC- |
| | | | | | | | specific |
| chr18:0-15,351,224 | p11.32-p11.21 | Loss | 3 | 2 | 1 | 0 | Mixed |
| chr22:16,205,534-27,894,332 | q11.1-q12.1 | Loss | 17 | 13 | 3 | 1 | Mixed |
| chr22:27,894,333-51,304,566 | q12.1-q13.33 | Loss | 18 | 14 | 3 | 1 | Mixed |

### EXAMPLE 5

Confirmation of Clinicopathological Importance of Changes in Recurrent Mutations and Copy Number Variation

As shown in [Table 1], mutations in BRAF and the TERT promoter were found more frequently in patients aged 45 years or older (p=0.031, p=0.002). The mutation in RAS was found more frequently in patients with bone metastasis (p<0.001). The mutation in the PLEKHS1 promoter was more common in cases of radioactive iodine therapy resistance (p=0.003).

With respect to the copy number variation, 11q gain was associated with bone metastasis (p=0.034) and cancer-specific death (p=0.028). The mutation in BRAF was associated with 1q gain (p=0.005) and 9q loss (p=0.002) (Table 8). The 9q loss was more common in patients who exhibited resistance to radioactive iodine therapy (p=0.002). The following [Table 8] shows the correlation between copy number variation and clinicopathological variables in differentiated thyroid cancer accompanied by distant metastasis.

TABLE 8

| | | p- | | p- | 9q | p- | 11q | p- | 22q | p- |
|---|---|---|---|---|---|---|---|---|---|---|
| Features | 1q gain | value | 9p loss | value | loss | value | loss | value | loss | value |
| Frequency | 8 (16%) | | 6 (12%) | | 10 | | 7 (14%) | | 18 (35%) | |
| | | | | | (25%) | | | | | |
| Age of distant | | 0.322 | | 0.575 | | 0.653 | | 0.328 | | 0.464 |
| metastasis | | | | | | | | | | |
| <45 years old | 0 | | 0 | | 1 (13%) | | 0 | | 2 (22%) | |
| (n = 9) | | | | | | | | | | |
| ≥45 years old | 8 (19%) | | 6 (14%) | | 9 (28%) | | 7 (17%) | | 16 (38%) | |
| (n = 42) | | | | | | | | | | |
| Sex | | 0.419 | | 0.170 | | 1.000 | | 0.376 | | 0.010 |
| Female (n = 37) | 7 (19%) | | 6 (16%) | | 7 (19%) | | 4 (11%) | | 17 (46%) | |
| Male (n = 14) | 1 (7%) | | 0 | | 2 (14%) | | 3 (21%) | | 1 (7%) | |
| Diagnosis | | 0.329 | | 0.331 | | 0.176 | | 0.126 | | 0.727 |
| PTC (n = 41) | 8 (20%) | | 6 (15%) | | 9 (22%) | | 4 (10%) | | 14 (34%) | |
| Non-PTC (n = 10) | 0 | | 0 | | 0 | | 3 (30%) | | 4 (40%) | |
| Histological | | 0.249 | | 0.680 | | 0.136 | | 1.000 | | 0.123 |
| type | | | | | | | | | | |
| Non-aggressive | 3 (10%) | | 3 (10%) | | 3 (10%) | | 4 (13%) | | 8 (27%) | |
| (n = 33) | | | | | | | | | | |
| Aggressive (n = | 5 (24%) | | 3 (14%) | | 6 (29%) | | 3 (14%) | | 10 (48%) | |
| 18) | | | | | | | | | | |
| Distant | | | | | | | | | | |
| metastasis | | | | | | | | | | |
| Lungs (n = 48) | 7 (15%) | 0.407 | 5 (10%) | 0.319 | 8 (17%) | 0.449 | 6 (13%) | 0.364 | 16 (33%) | 0.282 |
| Bones (n = 17) | 4 (24%) | 0.416 | 1 (6%) | 0.650 | 4 (24%) | 0.459 | 5 (30%) | 0.034 | 8 (47%) | 0.214 |
| RAI | | 0.063 | | 1.000 | | 0.029 | | 0.216 | | 0.186 |
| Effective (n = 29) | 2 (7%) | | 3 (10%) | | 2 (7%) | | 2 (7%) | | 8 (28%) | |
| Resistant (n = 22) | 6 (28%) | | 3 (14%) | | 7 (32%) | | 5 (23%) | | 10 (46%) | |
| Survival state | | 0.234 | | 1.000 | | 0.284 | | 0.028 | | 0.652 |
| Survived (n = 45) | 6 (13%) | | 6 (13%) | | 7 (16%) | | 4 (9%) | | 15 (33%) | |
| Dead (n = 6) | 2 (33%) | | 0 | | 2 (33%) | | 3 (50%) | | 3 (50%) | |
| BRAF V600E | | 0.005 | | 0.671 | | 0.002 | | 0.693 | | 0.782 |
| Wild-type (n = 24) | 0 | | 2 (8%) | | 0 | | 4 (17%) | | 8 (33%) | |
| Mutant (n = 27) | 8 (30%) | | 4 (15%) | | 9 (33%) | | 3 (11%) | | 10 (37%) | |
| Mutation in | | 0.329 | | 0.331 | | 0.176 | | 1.000 | | 0.296 |
| RAS | | | | | | | | | | |
| Wild-type (n = 41) | 8 (20%) | | 6 (15%) | | 9 (22%) | | 6 (19%) | | 13 (32%) | |
| Mutant (n = 10) | 0 | | 0 | | 0 | | 1 (13%) | | 5 (50%) | |

According to a somatic CNA analysis of TCGA, the 1q gain is frequently found in tall cell variant papillary thyroid carcinoma and V600E mutation in BRAF, and is associated with a more aggressive papillary thyroid carcinoma morphology. The loss of 22q usually occurred in the follicular variant of papillary thyroid carcinoma. In the TCGA study, 1q gain and 22q loss were found in 13.6% and 11.2% of 250 patients with papillary thyroid carcinomas without metastasis, respectively. In the presence of distant metastasis, 1q gain was confirmed in 25% (8/32) of papillary thyroid carcinomas, and BRAF V600E was abundant, but was found not to be associated with tall cell variants. The loss of 22q was found in 38% (12/32) of papillary thyroid carcinomas and 43% (3/7) of follicular thyroid carcinomas, and is not associated with follicular growth patterns. In a study conducted by the MSKCC, 1q and 22q losses were found in 26% and 14% of 57 patients with aggressive differentiated thyroid cancer (35 poorly papillary thyroid carcinomas, 18 papillary thyroid carcinomas and 4 Hurthle cell carcinomas), respectively. In 18 cases of papillary thyroid carcinomas, 1q gain and 22q loss were confirmed to be 5 (28%) and 2 (11%), respectively. A difference in prevalence rate between 1q gain and 22q loss and clinicopathological characteristics among various studies including the present invention occur due to a limited number of cases and differences in study design. However, in the present study, the high frequency of 1q gain coincided with the results of a study on a fatal papillary thyroid carcinoma group conducted by the MSKCC. These results suggest that 1q gain plays an important role in imparting aggressiveness against differentiated thyroid cancer.

### EXAMPLE 6

Factors Associated with Radioactive Iodine Therapy Resistance

In a univariate analysis, clinical factors significantly associated with radioactive iodine therapy resistance were an age of 45 years or older (p=0.021) and bone metastasis (p<0.001). In order to improve the ability to predict radioactive iodine therapy responses in patients with differentiated thyroid cancer, the present inventors added a combination of recurrent mutations as an independent variable. The presence of mutations in the TERT promoter, PLEKHS1 promoter or TP53 was significantly associated with radioactive iodine therapy resistance (p=0.001). Sex and aggressive histological types were not greatly associated with radioactive iodine therapy resistance. In a multivariate logistic regression analysis, having any one of mutations in the TERT promoter or PLEKHS1 promoter or TP53 was still significantly associated with radioactive iodine therapy resistance (adjusted odds ratio=7.64, 95% confidence interval=1.78 to 32.76, p=0.006).

### EXAMPLE 7

Cancer-Specific Survival in Patients with Metastatic Differentiated Thyroid Cancer

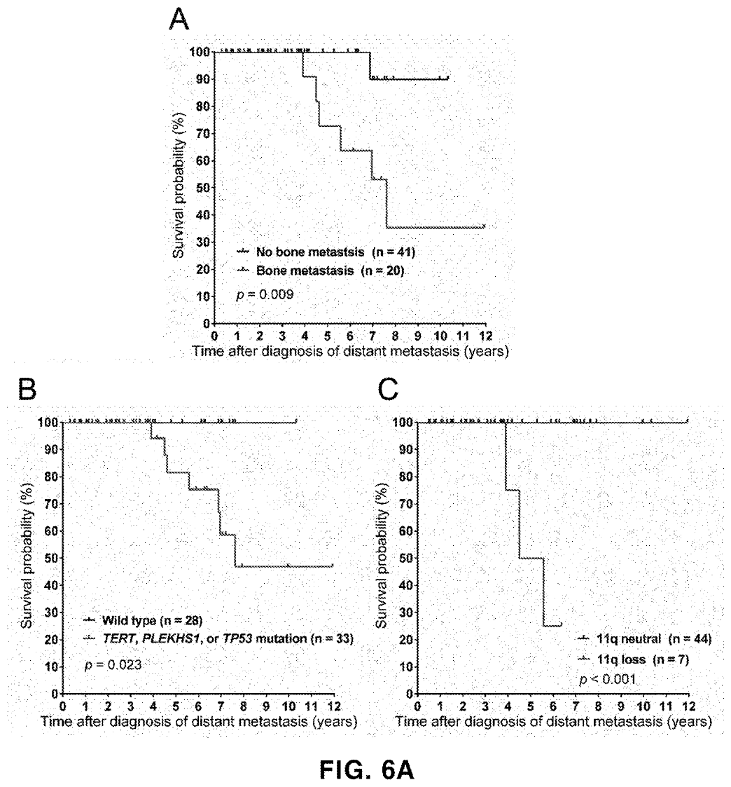

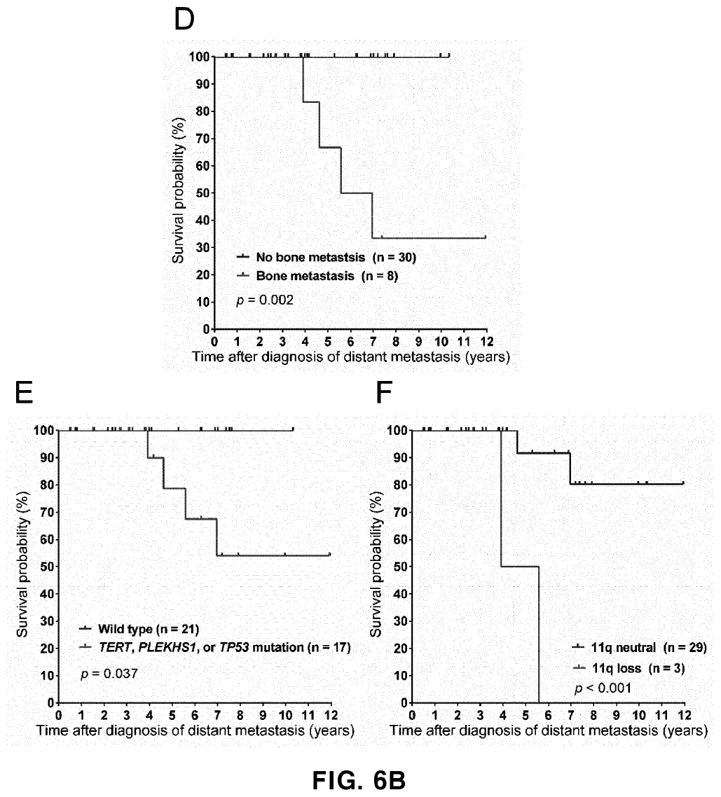

The survival rate of patients with radioactive iodine therapy resistance was significantly aggravated (p=0.005). Bone metastasis (p=0.009), the mutation (p=0.023) in at least one of the TERT promoter, the PLEKHS1 promoter or a TP53 gene and 11q loss copy number variation (p<0.001) were particularly associated with a poor cancer-specific survival (FIG. 6A, A to C). At the age of 45 or older in males (p=0.090), the cancer-specific survival was also aggravated, but was not statistically significant. There was no relationship between aggressive histological type and cancer-specific survival (data not shown).

When gene mutation types analyzed from 38 primary papillary thyroid carcinomas and cancer-specific survival were additionally analyzed, the poor cancer-specific survival was associated with bone metastasis (p=0.002), mutation (p=0.037) in at least one of the three high-risk genes, and 11q loss (p<0.001) (FIG. 6B, D to F).

### EXAMPLE 8

Genetic Classification and Risk Stratification of Metastatic Differentiated Thyroid Cancer

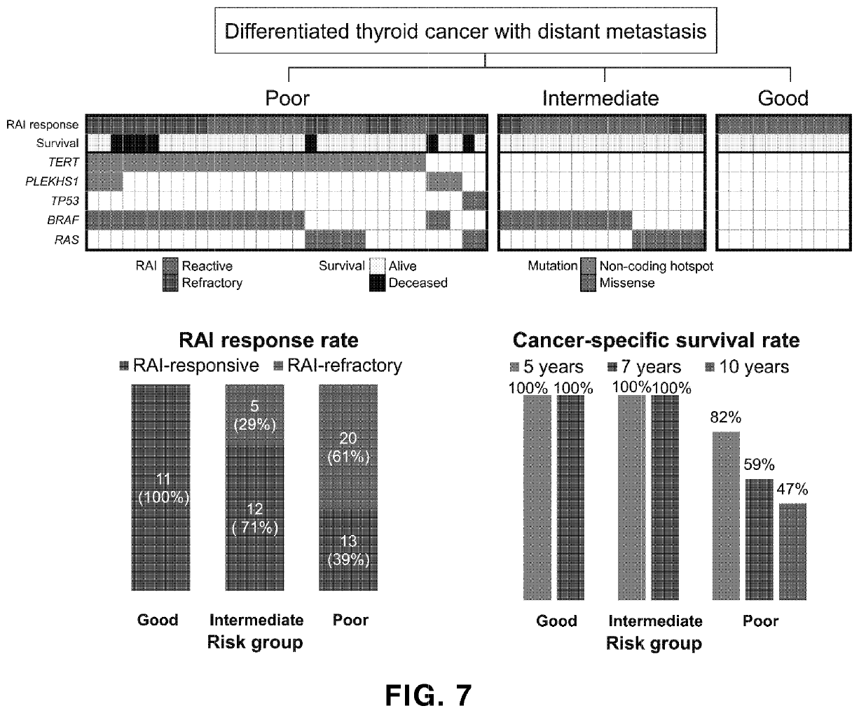

Using repeatedly mutated genes, the present inventors attempted to develop a genetic discriminator for predicting therapeutic response and prognosis based on the following principles; 1) selecting a mutation that occurs repeatedly from 3% or more of the total, 2) including a mutation that has already been well reported in the literature such as BRAF and RAS for basic molecular pathological classification and thus has a sufficient ground and a mutation in the TERT promoter and TP53, whose correlation with poor prognosis has already been clarified, and 3) including a mutation which is statistically clarified in the present invention to be associated with poor prognosis (distant metastasis, radioactive iodine therapy resistance, disease-specific survival). Based on the above principle, seven genes (BRAF, three types of RAS, a TERT promoter, a PLEKHS1 promoter and TP53) were selected. The loss of 11q was not selected because the copy number variation data was omitted from 10 samples. By analyzing the relationship between clinical results and mutation profiles of these genes, a new genetic discriminator capable of classifying patients with differentiated thyroid cancer with distant metastasis into three prognosis groups (FIG. 7) could be developed:

1) Poor prognosis group: 54% (33/61) of total patients with metastatic differentiated thyroid cancer were classified into a poor prognosis group, where the patients retained at least one or more mutations in the TERT promoter, the PLEKHS1 promoter or TP53 regardless of the mutation state of BRAF and RAS. In this group, a radioactive iodine therapy resistance rate was 61% (20/33) and mortality due to thyroid cancer during the follow-up period was 21% (7/33). The 5-year, 7-year, and 10-year cancer-specific survival rates were 82%, 59%, and 47%, respectively.

2) Intermediate prognosis group: 28% (17/61) of total patients with metastatic differentiated thyroid cancer were classified into an intermediate prognosis group, where the patients retained a mutation in a BRAF or RAS gene, but did not have a mutation in the TERT promoter, the PLEKHS1 promoter or TP53. This group had a radioactive iodine therapy resistance rate of 29% (5/17) and did not die from thyroid cancer.

3) Good prognosis group: This group corresponded to 18% (11/61) of total patients, and the patients are free of mutations in seven major genes. All patients in this group responded to radioactive iodine therapy and none died.

### EXAMPLE 9

Mutation Profile

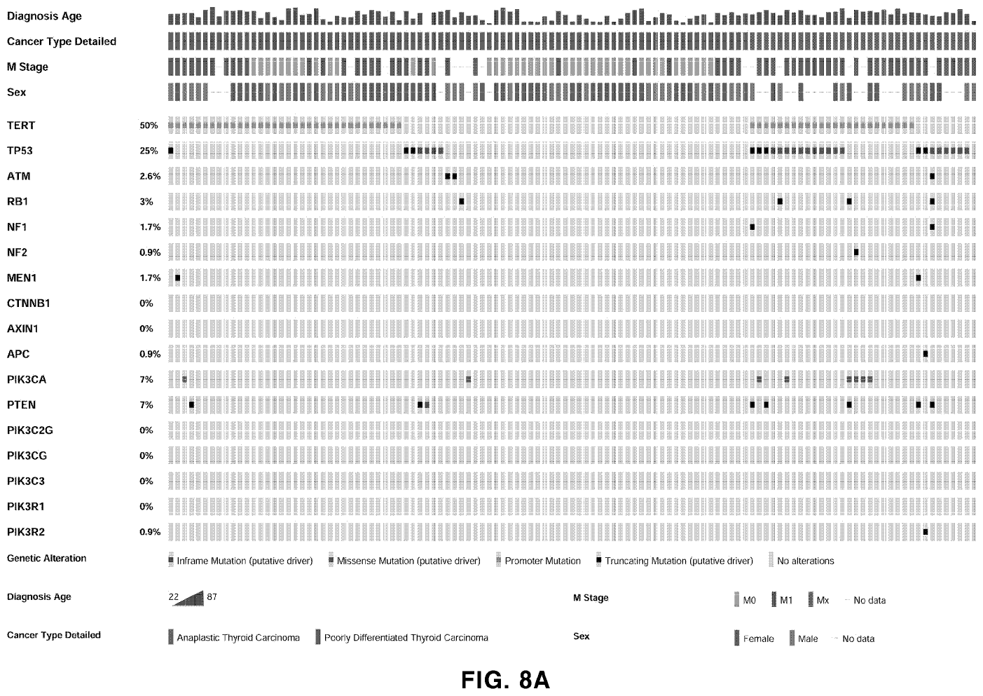

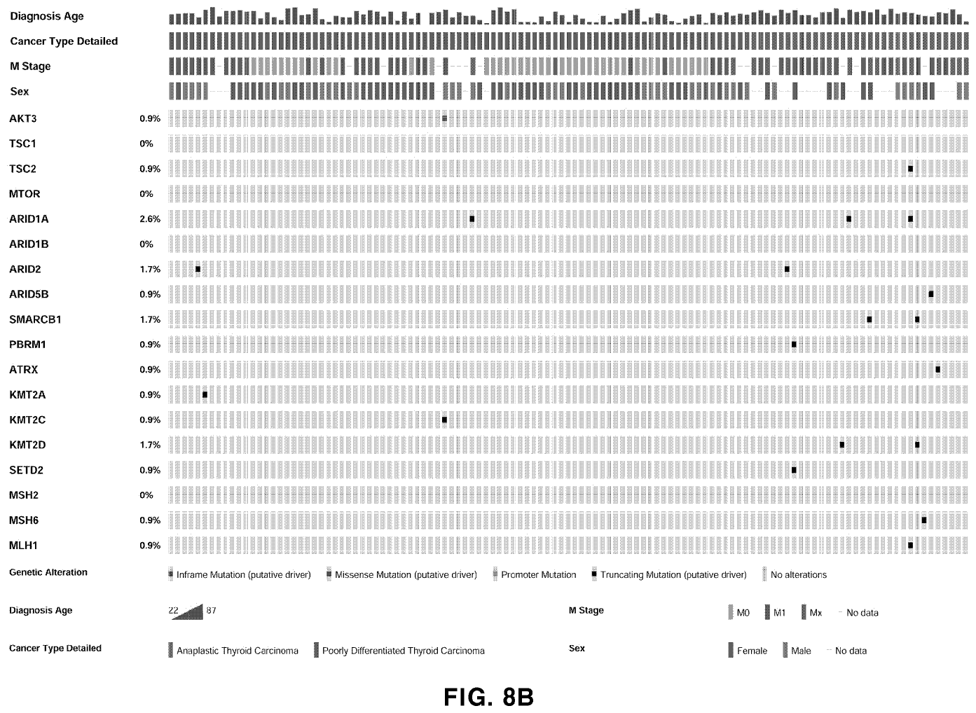

Gene mutation types were analyzed using cBioPortal (http://cbioportal.org) for cancer genomes of patients (n=33) with anaplastic thyroid carcinomas and patients (n=84) with poorly differentiated thyroid carcinomas from the Memorial Sloan Kettering Cancer Center (MSKCC). The mutations in the TERT promoter, TP53 and the PIK3CA gene were shown to be 50%, 25% and 7%, respectively. Seven of the eight carcinomas with mutations in PIK3CA were accompanied by a mutation in the TERT promoter, two carcinomas were accompanied by mutations in the TERT promoter and the TP53 gene, and no mutation in AKT1 was found. Eight carcinomas with the mutation in PIK3CA simultaneously showed the mutation in the TERT promoter. Proportions of mutations in other genes were shown to be as low as 0 to 7%, and appeared almost simultaneously with the mutation in the TERT promoter or TP53 (FIGS. 8A and 8B). In another study conducted at the MD Anderson Cancer Center, a mutation in PIK3CA was found in 6 of 190 advanced differentiated thyroid cancer cases with distant metastasis or a persistent/recurrent local disease. Of the six tumors with the mutation in PIK3CA, one was found together with the mutation in TP53.

In the present invention, 33 (54%) out of 61 cases had a mutation in at least one of the TERT promoter, the PLEKHS1 promoter or the TP53 gene. The mutation in PIK3CA was simultaneously found together with the mutation in the PLEKHS1 promoter in one case. Other rare mutations associated with advanced thyroid cancer were found mostly together with mutations in three genes.

Therefore, it is determined that the genetic discriminator of the present invention based on the mutation in the TERT promoter, the PLEKHS1 promoter, or TP53 can improve the clinical usefulness of other recurrent gene mutations including the mutations in PIK3CA and AKT1 in predicting the therapeutic results of patients with differentiated thyroid cancer.

Statistical Analysis

A gene result analysis was performed without knowing the clinicopathological data, and a usefulness analysis of the clinicopathological factors was performed without knowing the gene result. The correlation between clinicopathological factors and gene mutation types was performed by the hi-squared test for a parametric test and the Fisher's exact test for a non-parametric test. A logistic regression analysis was performed to determine whether clinicopathological factors and mutations are associated with radioactive iodine therapy responses. A disease-specific survival curve was drawn by the Kaplan-Meier method, and the difference between the curves was statistically verified using the log-rank test. Disease-specific survival was defined from the time of diagnosis of distant metastasis to the time of death from thyroid cancer or the time of final follow-up observation. All statistical analyses were performed using Prism (version 6.05, GraphPad Software, La Jolla, Calif., USA) and SPSS (version 21.0, IBM Corp, Armonk, N.Y., USA) statistical programs. Cases where the P statistical value was less than 0.05 were determined to be statistically significant.

The biomarker composition for diagnosing or predicting the prognosis of thyroid cancer of the present invention confirms whether a mutation is present in a PLEKHS1 promoter gene, and thus can provide information needed for diagnosing metastatic (distant metastatic) differentiated thyroid cancer, and also confirms whether a mutation is present in BRAF, TERT promoter, three types of RAS and a TP53 gene in addition to the PLEKHS1 promoter gene, and thus, with respect to radioactive iodine therapy response and survival, can classify the prognosis of a metastatic differentiated thyroid cancer patient into one of three prognosis groups, and predict the same, and thus is highly industrially applicable. [Sequence Listing Free Text]

SEQ ID NO: 1 is a base sequence of a primer (forward) for digital PCR for verifying PLEKHS1 G590A.

SEQ ID NO: 2 is a base sequence of a primer (reverse) for digital PCR for verifying PLEKHS1 G590A.

SEQ ID NO: 3 is a base sequence of a probe for digital PCR for verifying PLEKHS1 G590A.

SEQ ID NO: 4 is a base sequence of a primer (forward) for digital PCR for verifying PLEKHS1 C593T.

SEQ ID NO: 5 is a base sequence of a primer (reverse) for digital PCR for verifying PLEKHS1 C593T.

SEQ ID NO: 6 is a base sequence of a probe for digital PCR for verifying PLEKHS1 C593T.

SEQ ID NO: 7 is a base sequence of a first primer (forward) for Sanger sequencing for verifying TERT.

SEQ ID NO: 8 is a base sequence of a first primer (reverse) for Sanger sequencing for verifying TERT.

SEQ ID NO: 9 is a base sequence of a second primer (forward) for Sanger sequencing for verifying TERT.

SEQ ID NO: 10 is a base sequence of a second primer (reverse) of Sanger sequencing for verifying TERT.

SEQ ID NO: 11 is a base sequence of a third primer (forward) for Sanger sequencing for verifying TERT.

SEQ ID NO: 12 is a base sequence of a third primer (reverse) for Sanger sequencing for verifying TERT.

SEQ ID NO: 13 is a base sequence of a primer (forward) for Sanger sequencing for verifying BRAF exon 15.

SEQ ID NO: 14 is a base sequence of a primer (reverse) for Sanger sequencing for verifying BRAF exon 15.

SEQ ID NO: 15 is a base sequence of a primer (forward) for Sanger sequencing for verifying NRAS codon 61.

SEQ ID NO: 16 is a base sequence of a primer (reverse) for Sanger sequencing for verifying NRAS codon 61.

SEQ ID NO: 17 is a base sequence of a primer (forward) for Sanger sequencing for verifying NRAS codons 12 and 13.

SEQ ID NO: 18 is a base sequence of a primer (reverse) for Sanger sequencing for verifying NRAS codons 12 and 13.

SEQ ID NO: 19 is a base sequence of a primer (forward) for Sanger sequencing for verifying HRAS codon 61.

SEQ ID NO: 20 is a base sequence of a primer (reverse) for Sanger sequencing for verifying HRAS codon 61.

SEQ ID NO: 21 is a base sequence of a primer (forward) for Sanger sequencing for verifying HRAS codons 12 and 13.

SEQ ID NO: 22 is a base sequence of a primer (reverse) for Sanger sequencing for verifying HRAS codons 12 and 13.

SEQ ID NO: 23 is a base sequence of a first primer (forward) for Sanger sequencing for verifying KRAS codon 61.

SEQ ID NO: 24 is a base sequence of a first primer (reverse) for Sanger sequencing for verifying KRAS codon 61.

SEQ ID NO: 25 is a base sequence of a second primer (forward) for Sanger sequencing for verifying KRAS codon 61.

SEQ ID NO: 26 is a base sequence of a second primer (reverse) for Sanger sequencing for verifying KRAS codon 61.

SEQ ID NO: 27 is a base sequence of a primer (forward) for Sanger sequencing for verifying KRAS codons 12 and 13.

SEQ ID NO: 28 is a base sequence of a primer (reverse) for Sanger sequencing for verifying KRAS codons 12 and 13.

SEQ ID NO: 29 is a base sequence of a primer (forward) for Sanger sequencing for verifying STK11 exon1.

SEQ ID NO: 30 is a base sequence of a primer (reverse) for Sanger sequencing for verifying STK11 exon1.

SEQ ID NO: 31 is a base sequence of a primer (forward) for Sanger sequencing for verifying STK11 exon2.

SEQ ID NO: 32 is a base sequence of a primer (reverse) for Sanger sequencing for verifying STK11 exon2.

SEQ ID NO: 33 is a base sequence of a primer (forward) for Sanger sequencing for verifying STK11 exon3.

SEQ ID NO: 34 is a base sequence of a primer (reverse) for Sanger sequencing for verifying STK11 exon3.

SEQ ID NO: 35 is a base sequence of a primer (forward) for Sanger sequencing for verifying STK11 exon4 & 5.

SEQ ID NO: 36 is a base sequence of a primer (reverse) for Sanger sequencing for verifying STK11 exon4 & 5.

SEQ ID NO: 37 is a base sequence of a primer (forward) for Sanger sequencing for verifying STK11 exon6.

SEQ ID NO: 38 is a base sequence of a primer (reverse) for Sanger sequencing for verifying STK11 exon6.

SEQ ID NO: 39 is a base sequence of a primer (forward) for Sanger sequencing for verifying STK11 exon7.

SEQ ID NO: 40 is a base sequence of a primer (reverse) for Sanger sequencing for verifying STK11 exon7.

SEQ ID NO: 41 is a base sequence of a primer (forward) for Sanger sequencing for verifying STK11 exon8.

SEQ ID NO: 42 is a base sequence of a primer (reverse) for Sanger sequencing for verifying STK11 exon8.

SEQ ID NO: 43 is a base sequence of a primer (forward) for Sanger sequencing for verifying STK11 exon9.

SEQ ID NO: 44 is a base sequence of a primer (reverse) for Sanger sequencing for verifying STK11 exon9.

SEQ ID NO: 45 is a base sequence of a primer (forward) for Sanger sequencing for verifying STK11 exon10a.

SEQ ID NO: 46 is a base sequence of a primer (reverse) for Sanger sequencing for verifying STK11 exon10a.

SEQ ID NO: 47 is a base sequence of a primer (forward) for Sanger sequencing for verifying STK11 exon10b.

SEQ ID NO: 48 is a base sequence of a primer (reverse) for Sanger sequencing for verifying STK11 exon10b.

SEQ ID NO: 49 is a base sequence of a primer (forward) for Sanger sequencing for verifying TP53 Exons 2, 3 and 4.

SEQ ID NO: 50 is a base sequence of a primer (reverse) for Sanger sequencing for verifying TP53 Exons 2, 3 and 4.

SEQ ID NO: 51 is a base sequence of a primer (forward) for Sanger sequencing for verifying TP53 Exons 2 and 3.

SEQ ID NO: 52 is a base sequence of a primer (reverse) for Sanger sequencing for verifying TP53 Exons 2 and 3.

SEQ ID NO: 53 is a base sequence of a primer (forward) for Sanger sequencing for verifying TP53 Exon 4.

SEQ ID NO: 54 is a base sequence of a primer (reverse) for Sanger sequencing for verifying TP53 Exon 4.

SEQ ID NO: 55 is a base sequence of a primer (forward) for Sanger sequencing for verifying TP53 Exons 5 and 6.

SEQ ID NO: 56 is a base sequence of a primer (reverse) for Sanger sequencing for verifying TP53 Exons 5 and 6.

SEQ ID NO: 57 is a base sequence of a primer (forward) for Sanger sequencing for verifying TP53 Exon 7.

SEQ ID NO: 58 is a base sequence of a primer (reverse) for Sanger sequencing for verifying TP53 Exon 7.

SEQ ID NO: 59 is a base sequence of a primer (forward) for Sanger sequencing for verifying TP53 Exons 8 and 9.

SEQ ID NO: 60 is a base sequence of a primer (reverse) for Sanger sequencing for verifying TP53 Exons 8 and 9.

SEQ ID NO: 61 is a base sequence of a primer (forward) for Sanger sequencing for verifying TP53 Exon 10.

SEQ ID NO: 62 is a base sequence of a primer (reverse) for Sanger sequencing for verifying TP53 Exon 10.

SEQ ID NO: 63 is a base sequence of a primer (forward) for Sanger sequencing for verifying TP53 Exon 11.

SEQ ID NO: 64 is a base sequence of a primer (reverse) for Sanger sequencing for verifying TP53 Exon 11.

SEQ ID NO: 65 is a base sequence of a primer (forward) for Sanger sequencing for verifying TP53_cDNA_1.

SEQ ID NO: 66 is a base sequence of a primer (reverse) for Sanger sequencing for verifying TP53_cDNA_1.

SEQ ID NO: 67 is a base sequence of a primer (forward) for Sanger sequencing for verifying TP53_cDNA_2.

SEQ ID NO: 68 is a base sequence of a primer (reverse) for Sanger sequencing for verifying TP53_cDNA_2.

SEQ ID NO: 69 is a base sequence of a primer (forward) for Sanger sequencing for verifying TP53_cDNA_3.

SEQ ID NO: 70 is a base sequence of a primer (reverse) for Sanger sequencing for verifying TP53_cDNA_3.

1. A biomarker composition for diagnosing or predicting a prognosis of thyroid cancer, comprising a preparation capable of detecting a mutation in a PLEKHS1 promoter gene.

2. The biomarker composition of claim 1, further comprising a preparation capable of detecting a mutation in any one or more genes selected from the group consisting of a TERT promoter gene, a TP53 gene, an STK11 gene, a BRAF gene and an RAS gene.

3. The biomarker composition of claim 1, wherein the prognosis is likely to be resistance to radioactive iodine therapy or death.

4. The biomarker composition of claim 1, wherein the diagnosis is differentiated thyroid cancer with distant metastasis.

5. The biomarker composition of claim 1, wherein the thyroid cancer is any one or more selected from the group consisting of a papillary thyroid carcinoma (PTC), a follicular thyroid carcinoma (FTC) and a poorly differentiated thyroid carcinoma (PDTC).

6. The biomarker composition of claim 1, further comprising a preparation capable of confirming a copy number variation of a loss of the long arm (q) of chromosome 22.

7. The biomarker composition of claim 6, wherein the loss of the long arm (q) of chromosome 22 is a loss of q11.1-q13.33 regions of chromosome 22.

8. The biomarker composition of claim 1, further comprising a preparation capable of confirming any one or more copy number variations selected from the group consisting of
a) gain of the long arm (q) of chromosome 1;
b) loss of the short arm (p) of chromosome 9;
c) loss of the long arm (q) of chromosome 9; and
d) loss of the long arm (q) of chromosome 11.

9. The biomarker composition of claim 8, wherein
a) the gain of the long arm (q) of chromosome 1 is a gain of q12-q44 regions of chromosome 1;
b) the loss of the short arm (p) of chromosome 9 is a loss of p24.3-p13.1 and p13.1-p11.2 regions of chromosome 9;
c) the loss of the long arm (q) of chromosome 9 is a loss of q12-q31.1 and g31.1-q34.3 regions of chromosome 9; and
d) the loss of the long arm (q) of chromosome 11 is a loss of q11.2-q22.1, q22.1-q23.2 and q23.2-q24.3 regions of chromosome 11.

10. A kit for diagnosing or predicting a prognosis of thyroid cancer, comprising the biomarker composition of claim 1.

11. A method for providing information for diagnosing or predicting a prognosis of thyroid cancer, the method comprising: a) obtaining a gene from a sample isolated from an individual;
b) confirming whether a mutation is present in any one or more genes selected from the group consisting of a PLEKHS1 promoter gene, a TERT promoter gene and a TP53 gene in the gene of Step a); and
c) confirming whether a mutation is present in any one or more genes selected from the group consisting of a BRAF gene and a RAS gene when no mutation is found in Step b).

12. The method of claim 11, wherein the sample in Step a) is any one or more selected from the group consisting of tumor, blood, urine and saliva.

13. The method of claim 11, wherein, when the mutation in Step b) is confirmed, the individual is determined to be likely to exhibit resistance to radioactive iodine therapy and to die.

14. The method of claim 11, wherein, when the mutation in Step c) occurs, the individual is determined to exhibit resistance to radioactive iodine therapy, and when the mutation is not found, the individual is determined to show a good prognosis.

15. The method of claim 11, wherein, in Step b), the copy number variations of any one or more selected from the group consisting of,
among the genes isolated in Step a),
a) gain of the long arm (q) of chromosome 1;
b) loss of the long arm (q) of chromosome 9;
c) loss of the long arm (q) of chromosome 11; and
d) loss of the long arm (q) of chromosome 22 are further confirmed.

16. The method of claim 15, wherein, when the copy number variation is confirmed, a patient with differentiated thyroid cancer is determined to be likely to exhibit resistance to radioactive iodine therapy and to die.
